(19) United States
(12) Patent Application Publication
(10) Pub. No.: US 2005/0238693 A1
(43) Pub. Date: Oct. 27, 2005

(54) Process for the preparation and activation of susbstances and a means of producing same

(75) Inventor: Susan Kay Whyte, Dalkeith Wa (AU)

Correspondence Address: MCKENNA LONG & ALDRIDGE LLP, 1900 K STREET, NW, WASHINGTON, DC, 20006, US

(73) Assignee: Chemstop Pty Ltd., Dalkeith (AU)

(21) Appl. No.: 10/514,992

(22) PCT Filed: May 20, 2003
(86) PCT No.: PCT/AU03/00607

§ 371 Date: Nov. 19, 2004

(30) Foreign Application Priority Data
May 20, 2002 (AU) PS 2400
May 22, 2002 (AU) PS 2480

(51) Int. Cl. A61N 1/00; A61K 38/00; A61K 48/00; A01N 43/04; A01N 25/00; A61K 47/00

(52) U.S. Cl. 424/439; 504/116.1; 514/2; 514/44; 607/1

(57) ABSTRACT

The present invention relates to a process for the preparation and activation of a substance and a means for producing the activated substance. In particular, the invention relates to a method of treating a disease in a subject in need of such treatment, comprising the step of administering a substance or active agent which comprises one or more components which have been agitated such that a harmonic of between 20 to 50 Hz has been produced, in an amount effective to treat said disease, with the proviso that the disease is not an airway disorder.

### FIELD OF THE INVENTION

The present invention relates to a process for the preparation and activation of a substance and a means for producing the activated substance. In particular, the invention relates to a process of preparing a substance, wherein the substance is activated such that the efficacy and/or bioavailability of the substance is increased by agitating the substance or one or more components of the substance so that a specific harmonic is obtained. In one embodiment the activated substance is capable of regulating the cytochrome P₄₅₀ pathways and thereby overcoming or at least alleviating conditions associated with reactive oxygen species (ROS).

### BACKGROUND OF THE INVENTION

It is well appreciated by those skilled in the art that many substances including food, therapeutics, agricultural chemicals including pesticides, herbicides, and other industrial chemicals have limited efficacy in use. This is despite these substances having been used, in some instances, for thousands of years. Many foodstuffs, for example, are known to be poorly digested and/or absorbed by the gastrointestinal tract. Also the efficacy and/or bioavailability of therapeutics have often proven disappointing even though these materials have proven useful in in vitro systems. More concerning is that certain foodstuff and therapeutics may even have harmful effects attributed to them.

In an attempt to overcome or at least alleviate some of these problems a number of researchers have worked on improving particular substances or modifying the biological systems being affected. One area that has received some attention recently has been the role of the cytochrome P₄₅₀ enzyme system and in particular the protective effects this system provides against reactive oxygen species (ROS).

The production of ROS, including free radicals and free radical products is known to be deleterious. Further, ROS are also produced by one-electron peroxidase oxidations to cation radicals, stabilisation of the ROS generator, CYP2E1. ROS are known to be cytotoxic and cause inflammatory disease, including tissue necrosis, arthritis and deficits in energy metabolism (Manual et al, 2000).

Free radicals are formed in the body through unpaired electrons formed when there is no apparent enzyme synthesised by the liver to match the corresponding electron of an atom of certain substances in the body. These substances are often particles of synthetic chemical compounds for which the human enzyme system has not yet developed enzymes to enable complete detoxification via the liver and excretory organs, for example the bowel, kidneys and skin. The free radicals formed in this manner roam free in the body and contribute to inflammation and other harmful cellular changes in a variety of tissues, for example in the tendons, muscles, ligaments and bones (Lall et al, Indian Journal of Experimental Biology. 37 (2): 109-16, February 1999) Demineralisation is also considered to contribute to free radical pathology (Lall et al, supra); thus resulting in arthritis, inflammatory joint and soft tissue disease and osteoporosis.

Trace elements including zinc, magnesium and selenium are some of the elements involved in antioxidant defence mechanisms. Inadequate intake of these nutrients have been associated with ischemic heart disease, arthritis, stroke and cancer, where pathogenic role of free radicals is suggested (Lall et al, supra).

Whilst certain vitamin and mineral supplements are known, as are specific treatments for the remedy of certain of the medical conditions mediated by free radicals and ROS, there is no formulation available capable of preventing and treating effectively a wide range of medical conditions mediated by free radicals.

Further, the role played by nutrition in protecting against the effects of ROS have only recently been acknowledged. The biological antioxidant defence system includes glutathione reductase, glutathione-s-transferase, glutathione peroxidase, phospholipid hydroperoxide glutathione peroxidase, superoxide dismutase (SOD) which is a selenium dependent enzyme and catalase, together with the antioxidant vitamins C and E. The individual components of this system are utilised in various physiological and protective processes and therefore require replenishment from the diet. Other components of the diet including carbohydrates, proteins and lipids are known to be important for maintaining the levels of various enzymes required in body's defence system providing protection against toxins for example heavy metals such as lead which can contribute to loss of bone density (Zerwekh and Pak, 1998).

Accordingly, there is a need to improve a range of substances so that increased efficiency, efficaciousness and/or bioavailability is produced. Also there is a need to provide foodstuff and therapeutics that are capable of regulating the cytochrome P₄₅₀ pathways such that the host defence mechanisms are able to counteract the effects of ROS.

The applicant has now surprisingly found that substances, may be enhanced with respect to efficacy and/or bioavailability by using specific agitation methods which produce particular harmonics.

Moreover, certain substances produced by the methods of the present invention are capable of regulating the cytochrome P₄₅₀ pathways allowing the effective prevention and/or treatment of disorders mediated at least in part by free radicals.

### SUMMARY OF THE INVENTION

A first aspect of the invention provides an active substance, wherein said substance has been agitated such that a harmonic of between 20 to 50 Hz has been produced.

A second aspect of the invention provides a process of preparing an active substance comprising the step of agitating said substance such that a harmonic of between 20 to 50 Hz is produced.

A third aspect of the invention provides a device for preparing an active substance comprising a container and a agitator, wherein said device is capable of producing in a substance a harmonic of between 20 to 50 Hz.

A fourth aspect of the invention provides a method of treating a disease in a subject in need of such treatment, comprising the step of administering a substance or active agent which comprises one or more components which have been agitated such that a harmonic of between 20 to 50 Hz has been produced, in an amount effective to treat said disease.

A fifth aspect of the invention provides a substance or active agent useful for treating a disease in a subject in need of such treatment, comprising ascorbic acid, magnesium and selenomethionine and a pharmaceutically acceptable carrier, wherein at least one component has been agitated such that a harmonic of between 20 to 50 Hz has been produced, together in an amount effective to treat said disease.

A sixth aspect of the invention provides a method of producing a substance or active agent useful for treating a disease in a subject in need of such treatment, said formulation or composition comprising vitamins, trace elements and probiotic bacteria said method comprising the step of agitating at least one component of said substance or active agent such that a harmonic of between 20 to 50 Hz is produced.

The foregoing and other aspects of the present invention are explained in greater detail in the specification below.

### BRIEF DESCRIPTION OF THE FIGURES

FIG. 1 shows energy of particles produced in a vortex in an isotropic.

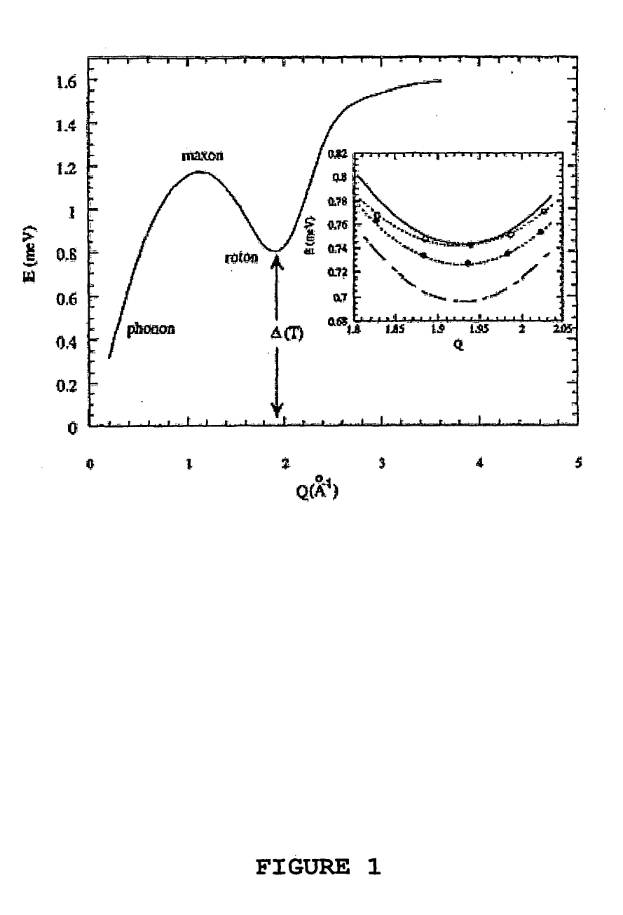

FIG. 2 shows energy gradient of produced rotons.

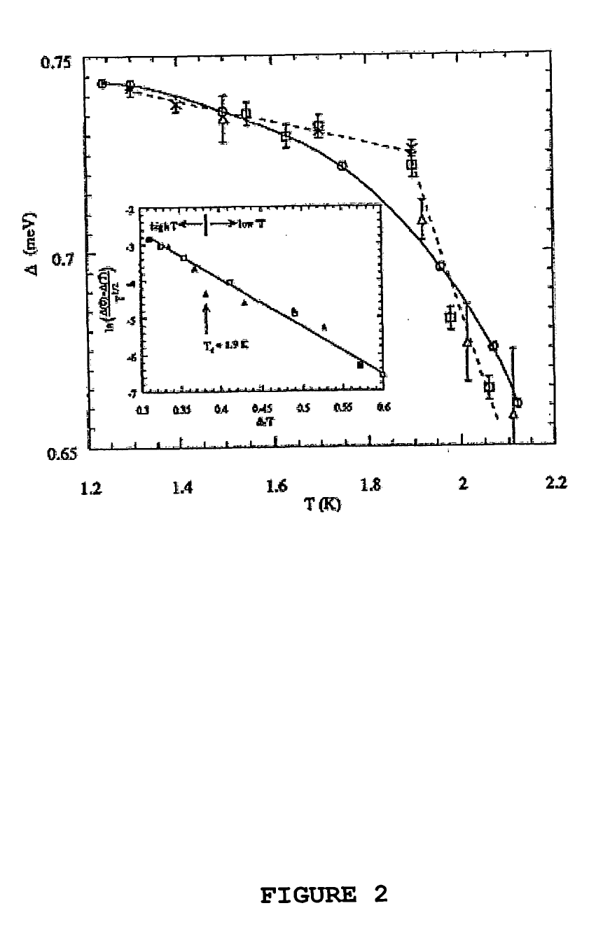

FIG. 3 shows the two competing processes of the apparatus contributing to the crossover behaviour in the roton energy.

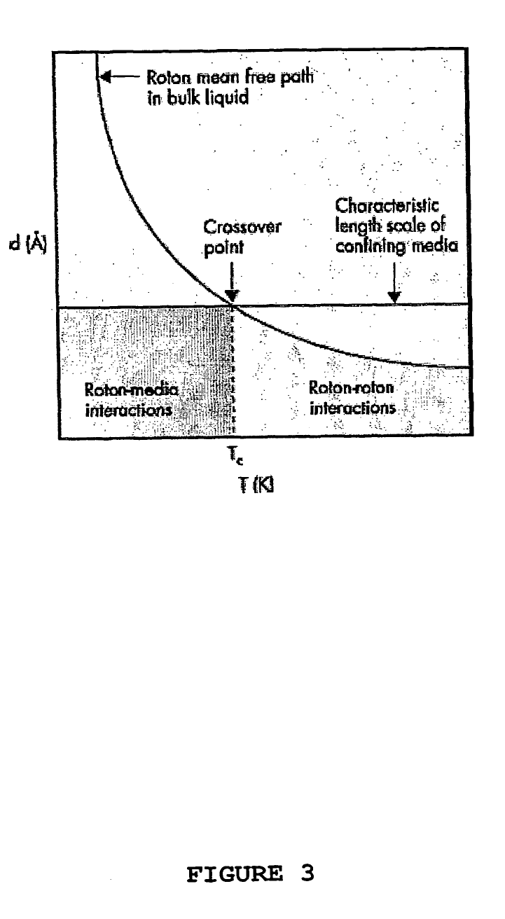

FIG. 4 shows a diagrammatic representation of process.

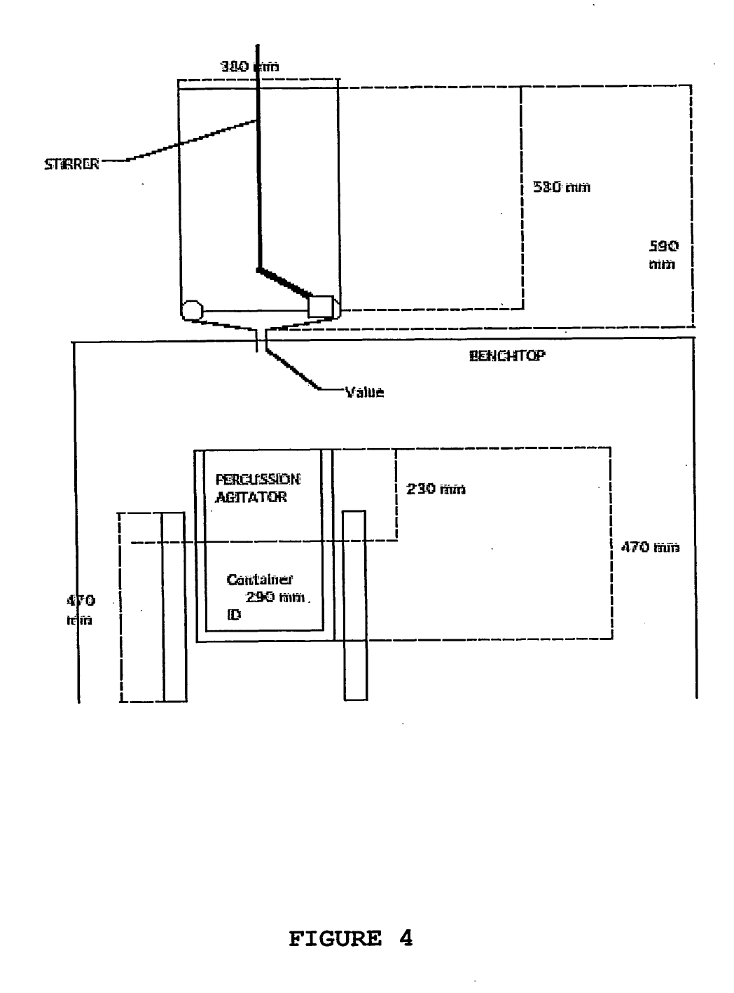

### DETAILED DESCRIPTION OF THE INVENTION

The practice of the present invention employs, unless otherwise indicated, conventional food production techniques, chemistry and pharmacology within the skill of the art. Such techniques are well known to the skilled worker, and are explained fully in the literature. See, eg., Coligan, Dunn, Ploegh, Speicher and Wingfield “Current protocols in Protein Science” (1999) Volume I and II (John Wiley & Sons Inc.); and Bailey, J. E. and Ollis, D. F., Biochemical Engineering Fundamentals, McGraw-Hill Book Company, NY, 1986.

Before the present methods are described, it is understood that this invention is not limited to the particular materials and methods described, as these may vary. It is also to be understood that the terminology used herein is for the purpose of describing particular embodiments only, and is not intended to limit the scope of the present invention which will be limited only by the appended claims. It must be noted that as used herein and in the appended claims, the singular forms “a,” “an,” and “the” include plural reference unless the context clearly dictates otherwise. Thus, for example, a reference to “a substance” includes a plurality of such substances, and a reference to “an harmonic” is a reference to one or more harmonics, and so forth. Unless defined otherwise, all technical and scientific terms used herein have the same meanings as commonly understood by one of ordinary skill in the art to which this invention belongs. Although any materials and methods similar or equivalent to those described herein can be used to practice or test the present invention, the preferred materials and methods are now described.

All publications mentioned herein are cited for the purpose of describing and disclosing the protocols, reagents and vectors which are reported in the publications and which might be used in connection with the invention. Nothing herein is to be construed as an admission that the invention is not entitled to antedate such disclosure by virtue of prior invention.

The present invention relates to a process of activation. The terms “active” and “activation” as used herein with reference to the “substance” means the ability to produce a substance that has enhanced effects. For example, with respect to chemicals such as herbicides and pesticides, the term “activation” means that these are more efficacious in that they kill plants or pests more effectively than the comparable amount of unactivated herbicide or pesticide. Activation with respect to foodstuff and therapeutics means that they are more efficacious and/or bioavailable when compared to the same amount of unactivated foodstuff or therapeutic. In one embodiment the “activated” substance is capable of regulating the cytochrome P₄₅₀ pathways and thereby overcoming or at least alleviating conditions in a subject associated with reactive oxygen species (ROS).

The term “subject” as used herein refers to any animal or plant species. However, the term “subject” depends upon the substance of the invention being activated and its end use. For example, if the substance being activated is a herbicide then the “subject” is a plant. If the substance being activated is a pesticide then the “subject” is an invertebrate or vertebrate pest. Some of methods of the present invention are particularly useful in the treatment of warm-blooded vertebrates. Thus, in a preferred embodiment, the “subject” of the invention concerns mammals and birds.

In one preferred embodiment the present invention is concerned primarily with the treatment of human subjects, but can also be employed for the treatment of other mammalian subjects, such as dogs, cats, livestock, primates and horses, for veterinary purposes.

Thus, provided is the treatment of mammals such as humans, as well as those mammals of economical importance and/or social importance to humans, for instance, carnivores other than humans (such as cats and dogs), swine (pigs, hogs, and wild boars), ruminants (such as cattle, oxen, sheep, giraffes, deer, goats, bison, and camels), and horses. Also provided is the treatment of birds, including the treatment of those kinds of birds that are endangered, kept in zoos, as well as fowl, and more particularly domesticated fowl, eg., poultry, such as turkeys, chickens, ducks, geese, guinea fowl, and the like, as they are also of economical importance to humans. Thus, provided is the treatment of livestock, including, but not limited to, domesticated swine (pigs and hogs), ruminants, horses, poultry, and the like.

The term “substance” as used herein is any substance which can benefit from being activated. For example, a substance may be a foodstuff, a chemical or a component of a chemical or foodstuff. Preferably the substance includes an active agent. As used herein, the term “active agent” refers to an agent which possesses useful properties such as a therapeutic or prophylactic activity in vivo, or herbicidal or pesticidal activity, or nutritional property. The term “active agent” also includes other (non-active) substances, which may, for example, be administered together with or combined with the active agent to aid application and/or administration. Examples of suitable active agents include proteins, such as hormones, antigens, and growth factors; chemicals such as herbicides, pesticides, dyes, and anti-oxidants, vitamins and minerals; probiotic bacteria; nucleic acids; and smaller molecules, such as antibiotics, steroids, and decongestants.

The active agent can include organic molecules such as a drug, peptide, protein, carbohydrate (including monosaccharides, oligosaccharides, and polysaccharides), nucleoprotein, mucoprotein, lipoprotein, synthetic polypeptide or protein, or a small molecule linked to a protein, glycoprotein, steroid, nucleic acid (any form of DNA, including cDNA, or RNA, or a fragment thereof), nucleotide, nucleoside, oligonucleotides (including antisense oligonucleotides), gene, lipid, hormone, vitamin, including vitamin C and vitamin E, minerals and elements such as magnesium, selenium or combinations thereof.

Representative therapeutic active agents include antioxidants, chemotherapeutic agents, steroids (including retinoids), hormones, antibiotics, antivirals, antifungals, antiproliferatives, antihistamines, anticoagulants, antiphotoaging agents, melanotropic peptides, nonsteroidal and steroidal anti-inflammatory compounds. Other non-limiting examples of active agents include anti-infectives such as nitrofurazone, sodium propionate, antibiotics, including penicillin, tetracycline, oxytetracycline, chlorotetracycline, bacitracin, nystatin, streptomycin, neomycin, polymyxin, gramicidin, chloramphenicol, erythromycin, and azithromycin; sulfonamides, including sulfacetamide, sulfamethizole, sulfamethazine, sulfadiazine, sulfamerazine, and sulfisoxazole, and anti-virals including idoxuridine; antiallergenics such as antazoline, methapyritene, chlorpheniramine, pyrilamine prophenpyridamine, hydrocortisone, cortisone, hydrocortisone acetate, dexamethasone, dexamethasone 21-phosphate, fluocinolone, triamcinolone, medrysone, prednisolone, prednisolone 21-sodium succinate, and prednisolone acetate; desensitizing agents such as ragweed pollen antigens, hay fever pollen antigens, dust antigen and milk antigen; decongestants such as phenylephrine, naphazoline, and tetrahydrazoline; miotics and anticholinesterases such as pilocarpine, esperine salicylate, carbachol, diisopropyl fluorophosphate, phospholine iodide, and demecarium bromide; parasympatholytics such as atropine sulfate, cyclopentolate, homatropine, scopolamine, tropicamide, eucatropine, and hydroxyamphetamine; sympathomimetics such as epinephrine; sedatives and hypnotics such as pentobarbital sodium, phenobarbital, secobarbital sodium, codeine, (α-bromoisovaleryl)urea, carbromal; psychic energizers such as 3-(2-aminopropyl)indole acetate and 3-(2-aminobutyl)indole acetate; tranquilizers such as reserpine, chlorpromayline, and thiopropazate; androgenic steroids such as methyl-testosterone and fluorymesterone; estrogens such as estrone, 17-β-estradiol, ethinyl estradiol, and diethyl stilbestrol; progestational agents such as progesterone, megestrol, melengestrol, chlormadinone, ethisterone, norethynodrel, 19-norprogesterone, norethindrone, medroxyprogesterone and 17-β-hydroxy-progesterone; humoral agents such as the prostaglandins, for example PGE₁, PGE₂ and PGF₂; antipyretics such as aspirin, sodium salicylate, and salicylamide; antispasmodics such as atropine, methantheline, papaverine, and methscopolamine bromide; antimalarials such as the 4-aminoquinolines, 8-aminoquinolines, chloroquine, and pyrimethamine, antihistamines such as diphenhydramine, dimenhydrinate, tripelennamine, perphenazine, and chlorphenazine; cardioactive agents such as dibenzhydroflume thiazide, flumethiazide, chlorothiazide, and aminotrate; nutritional agents such as vitamins, natural and synthetic bioactive peptides and proteins, including growth factors, cell adhesion factors, cytokines, and biological response modifiers.

Representative herbicidal active agents include any active agent previously used as an agent for controlling or eradicating plants. Non-limiting examples of herbicides are 2,4-D (WEEDAR™); 2,4-DB; DCPA (Dacthal™); DSMA (ARSONATE™; EPTC (EPTAM™); EPTC (ERADICANE™); MCPA (RHONOX™); MCPB (THISTROL™); MSMA (ANSAR™); acetochlor (HARNESS™); acetochlor (SURPASS™); acifluorfen (BLAZER™); alachlor (LASSO™); ametryn (EVIK™); amitrole (AMITROL-T™); asulam (ASULOX™); atrazine (AATREX™); azafenidin (MILESTONE™); benefin (BALAN™); bensulfuron (LONDAX™); bensulide (PREFAR™); bentazon (BASAGRAN™); bromacil (HYVAR-X™); bromoxynil (BUCTRIL™); butylate (SUTAN™); carfentrazone-ethyl (AIM™); chloramben (AMIBEN™); chlorimuron-ethyl (CLASSIC™); chlorpropham (FURLOE™); chlorsulfuron (GLEAN™); clethodim (PRISM™); clethodim (SELECT™); clomazone (COMMAND™); clopyralid (STINGER™); cloransulam (FIRST-RATE™); cyanazine (BLADEX™); cycloate (RO-NEET™); cycloxydim (FOCUS™); desmedipham (BETANEX™); dicamba (BANVEL™); dichlobenil (CASORON™); diclofop (HOELON™); diethatyl (ANTOR™); difenzoquat (AVENGE™); diflufenzopyr (DISTINCT™); dimethenamid (FRONTIER™); diquat (DIQUAT™); diuron (KARMEX™); endothall (DESICATE™); ethalfluralin (CURBIT™); ethalfluralin (SONALAN™); ethametsulfuron (MUSTER™); ethofumesate (NORTRON™); fenoxaprop-ethyl (BUGLE™); fenoxaprop-ethyl (OPTION II™); fluazifop-P (FUSILADE DX™); flucarbazone-sodium (MKH 6562™); flufenacet (AXIOM™); flumetsulam (BROADSTRIKE™); flumiclorac (RESOURCE™); flumioxazin (V-53482™); fluometuron (COTORAN™); fluroxypyr (STARANE™); fomesafen (FLEXSTAR™); fomesafen (REFLEX™); glufosinate (RELY™); glyphosate (ROUNDUP™); halosulfuron (PERMIT, SEMPRA™); haloxyfop (GALANT™); hexazinone (VELPAR™); imazameth (CADRE™); imazamethabenz (ASSERT™); imazamox (RAPTOR™); imazaquin (SCEPTER™); imazethapyr (PURSUIT™); isoxaben (GALLERY™); isoxaflutole (BALANCE™); lactofen (COBRA™); linuron (LOROX™); methazole (PROBE™); metolachlor (DUAL™); metribuzin (LEXONE™); metribuzin (SENCOR™); metsulfuron (ALLY™); molinate (ORDRAN™); napropamide (DEVRINOL™); naptalam (ALANA™); nicosulfuron (ACCENT™); norflurazon (SOLICAM™); oryzalin (SURFLAN™); oxadiazon (RONSTAR™); oxasulfuron (DYNAM™); oxyfluorfen (GOAL™); paraquat (GRAMOXONE EXTRA™); pebulate (TILLAM™); pelargonic acid (SCYTHE™); pendimethalin (PENTAGON™); pendimethalin (PROWL™); phenmedipham (SPIN-AID™); picloram (TORDON™); primisulfuron (BEACON™); prodiamine (BARRICADE™); prometryn (CAPAROL™); pronamide (KERB™); propachlor (RAMROD™); propanil (STAMPEDE™); prosulfuron (PEAK™); pyrazon (PYRAMIN™); pyridate (LENTAGRAN™); pyridate (TOUGH™); pyrithiobac (STAPLE™); quinclorac (FACE™); quizalofop (ASSURE™); rimsulfuron (MATRIX, SHADEOUT™); sethoxydim (POAST™); siduron (TUPERSAN™); simazine (PRINCEP™); sulfentrazone (AUTHORITY™); sulfometuron (OUST™); sulfosate (TOUCHDOWN™); sulfosulfuron (MON™); tebuthiuron (SPIKE™); terbacil (SINBAR™); thiazopyr (VISOR, MANDATE™); thifensulfuron (PINNACLE™); thiobencarb (BOLERO™); tralkoxydim (ACHEIVE™); triallate (FAR-GO™); triasulfuron (AMBER™); tribenuron (EXPRESS™); triclopyr (GARLON™); triclopyr (GRANDSTAND™); trifluralin (TREFLANT™); triflusulfuron (UPBEET™) and vernolate (VERNAM™).

Representative pesticidal active agents include 1,2-Dichloropropane; 1-Naphthaleneacetamid; 1-Naphthylacetic Acid; 2,4,5-T Acid; 2,4,5-T Amine Salts; 2,4,5-T Esters; 2,4-D-Acid; 2,4-DB Butoxyethyl ES; 2,4-DB Dimethylamine; ABAMECTIN™; ACEPHATE™; ACIFLUOREN™; ACIFLUORFEN™; ACROLEIN™; ALACHLOR™; ALDICARB™; ALDOXYCARB™; ALDRIN™; AMETRYN™; AMINOCARB™; AMITRAZ™; AMITROLE™; ANCYMIDOL™; ANILAZINE™; Arsenic Acid; Asulam, Sodium; ATRAZINE™; AZIMSULFURON™; AZINPHOS-ME™; BARBAN™; BENALAXYL™; BENDIOCARB™; BENEFIN™; BENODANIL™; BENOMYL™; BENSULFURON ME™; BENSULIDE™; BENTAZON™; BIFENOX™; BIFENTHRIN™; BROMACIL™; Bromoxynil Butyrate; BROMOXYNIL™; OCTANOATE™; BUTACHLOR™; Butylate; CAPTAFOL™; CAPTAN™; CARBARYL™; CARBENDAZIM™; CARBOFURAN™; Carbon Disulfide; CARBOPHENOTHION™; CARBOXIN™; CDAA; CHLORAMBEN™; CHLORBROMURON™; CHLORDANE™; Chlordimeform; Chlordimeform HCl; CHLORETHOXYFOS™; CHLORIDAZON™; CHLOROBENZILATE™; CHLORONEB™; CHLOROPICRIN™; CHLOROTHALONIL™; CHLOROXURON™; CHLORPROPHAM™; CHLORPYRIFOS™; Chlorpyrifos-Methyl; CHLORSULFURON™; CHLOZOLINATE™; CINMETHYLIN™; CLOFENTEZINE™; CLOMAZONE™; CLOPYRALID™; CRYOLITE™; CYANAZINE™; CYCLOATE™; CYFLUTHRIN™; CYHALOTHRIN™; CYHEXATIN™; CYMOXANIL™; CYPERMETHRIN™; CYROMAZINET™; DAMINOZIDE™; DAZOMET™; DBCP™; DCNA DICLORAN™; DDD™; DDE™; DDT™; DEMETON™; DESMEDIPHAM™; DI-ALLATE™; DIAZINON™; DICAMBA™; DICHLQBENIL™; DICHLONE™; DICHLORMID; DICHLOROPROPENE; DICHLORPROP; DICHLORVOS; DICLOFOP-ME; DICOFOL; DICROTOPHOS; DIELDRIN; DIENOCHLOR; DIFLUBENZURON; DIMETHIPIN; DIMETHIRIMOL; DIMETHOATE; DIMETHYLARSINIC ACID; DINITRAMINE; DINOCAP; DINOSEB; DIOXACARB; DIPROPETRYN; DIQUAT DIBROMIDE; DISULFOTON; DIURON; DNOC; DODINE ACETATE SALT; DSMA; ENDOSULFAN; ENDOTHALL; ENDRIN; EPN; EPTC; ESFENVALERATE; ETHALFLURALIN; ETHEPHON; ETHOFUMESATE; ETHOPROP; ETHYLENE DIBROMIDE; ETRIDIAZOLE; FENAMINOSULF; FENAMIPHOS; FENARIMOL; FENBUTATIN OXIDE; FENFURAM; FENITROTHION; FENOPROP; FENOXAPROP-ET; FENOXYCARB; FENPROPATHRIN; FENSULFOTHION; FENTHION; FENURON; FENVALERATE; FERBAM; FLUAZIFOP-BUTYL; FLUAZIFOP-P-BUTYL; FLUCHLORALIN; FLUCYTHRINATE; FLUMETRALIN; FLUMETSULAM; FLUOMETURON; FLUPYRSULFURON METHYL; FLURIDONE; FLUSILAZOLE; FLUSILAZOLEH™; FLUSILAZOLE; FOMESAFEN; FONOFOS; FORMETANATE HCl; FOSAMINE AMMONIUM; FOSAMINE AMMONIUM; FOSETYL ALUMINUM; GLUFOSINATE-AMMONIUM; GLYPHOSATE; HALOXYFOP-METHYL; HEPTACHLOR; HEXACHLOROBENZENE; HEXAZINONE; HEXAZINONEhtm; HEXAZINONEtxt; HYDRAMETHYLNON; IMAZALIL;IMAZAPYR ACID; IMAZAQUIN ACID; IMAZETHAPYR; IPRODIONE; ISAZOFOS; ISOFENPHOS; ISOPROPALIN; ISOXABEN; LACTOFEN; LENACIL; LENACILhtm; LENACILtxt; LINDANE; LINURON; MALATHION; MALEIC HYDRAZIDE ACID; MANCOZEB; MANEB; MCPA; MCPB; MECOPROP; MEFLUIDIDE; MEPIQUAT CHLORIDE; METALAXYL; METALDEHYDE; METHAMIDOPHOS; METHAM SODIUM; METHAZOLE; METHIOCARB; METHOMYL; METHOXYCHLOR™; Methyl Bromide; Methyl Isothiocyanate; Methyl Parathion; METIRAM™; METOLACHLOR™; METRIBUZIN™; METSULFURON ME™; MEVINPHOS™; MEXACARBATE™; MIREX™; MOLINATE™; MONOCROTOPHOS™; MONOLINURON™; MONURON™; MSMA™; MYCLOBUTANIL™; NALED™; Naphthalene; Napropamide; Naptalam Sodium Salt; NEBURON™; NICOSULFURON™; NITRAPYRIN™; NITROFEN™; NORFLURAZON™; ORYZALIN™; OXADIAZON™; OXAMYL™; OXYCARBOXIN™; OXYDEMETON-ME; OXYFLUORFEN; PACLOBUTRAZOL™; PARAQUAT DICHLORIDE™; PARATHION™; PEBULATE™; PENDIMETHALIN™; Pentachlorophenol; Perfluidone; Perimiphos-Ethyl; PERMETHRIN™; PHENMEDIPHAM™; PHENTHOATE™; PHORATE™; PHOSALONE™; PHOSMET™; PHOSPHAMIDON™; PICLORAM™; PIPERALIN™; PIRIMICARB™; PIRIMIPHOS-ETHYL; PRIMISULFURON-METHYL; PROCHLORAZ; PROCYMIDONE; PRODIAMINE; PROFENOFOS; PROFLURALIN; PROMECARB™; PROMETON; PROMETRYN; PROPACHLOR; PROPAMOCARB HCL; PROPANIL; PROPARGITE™; PROPAZINE™; PROPHAM; PROPICONAZOLE; PROPOXUR; PROPYZAMIDE™; PYRETHRINS™; PYRITHIOBAC SODIUM; QUINOMETHIONATE™; QUINTOZENE; QUIZALOFOP-ET; RESMETHRIN; RIMSULFURON; ROTENONE™; SECBUMETON; SETHOXYDIM; SIDURON™; SIMAZINE™; SIMETRYN™; SODIUM CHLORATE; SULFOMETURON-ME; SULPROFOS; TAU-FLUVALINATE™; TCA-SODIUM; TEBUTHIURON; TEMEPHOS; TERBACIL; TERBUFOS; TERBUTRYN; TETRACHLORVINPHOS™; THIABENDAZOLE™; THIDIAZURON™; THIOBENCARB™; THIODICARB™; THIOPHANATE-ME™; THIRAM™; TOLCLOFOS-METHYL™; TOXAPHENE™; TRALOMETHRIN™; TRIADIMEFON™; TRIADIMENOL; TRIALLATE™; TRIASULFURON™; TRIBUFOS™; TRICHLORFON™; TRICHLORONAT™; TRICLOPYR™; TRICYCLAZOLE™; TRIDIPHANE™; TRIFLUMIZOLE™; TRIFLURALIN™; TRIFLUSULFURON METHYL™; TRIFORINE™; TRIMETHACARB™; VINCLOZOLINT™; ZINEB™ and ZIRAM™.

Plant protection agents within the concept of the present invention are understood to include insecticides, acaricides, nematicides, repellants, fungicides, herbicides, rodenticides, and mulluscicides, as well as growth promoters and inhibitors and synergists. The chemical origin of these active substances is not critical. They may originate from the most varied classes of chemical compounds. The only requirement is that they must be stable under the manufacturing conditions for the carrier/active substance combinations. Thus, compounds from, for example, the chemical classes of the chlorocarbons (lindane and others), organophosphorus acid esters (parathion and others), carbamates (carbofuran and others), cyclodiene derivatives (endosulfan and others), pyrethroides, pyrethrins (cypermethrin and others), xanthogenates (dixanthogen and others), triazole derivatives (azocyclotin and others), organic sulfides (chlorfen sulfide and others), metal-organic compounds (cyhexatin and others), thiadiazine derivates (dazomet and others), phthalates (dimethylphthalate and others), morpholine derivatives (aldimorph and others), triazine derivatives (desmetryn and others), anilides (benodanil and others) imidazoles (benomyl and others), phthalimide derivatives (captan and others), sulfamides (dichlofluanid and others), pyrimidine derivatives (dimethirimol and others), thiadiazols (etridiazol and others), polymeric dithiocarbamates (maneb and others), monomeric dithiocarbamates (sulfallate and others), oxazolidine derivatives (vinchlozolin and others), urea derivatives (monolinuron and others), benzoic acid derivatives (chlorothiamid, dichlobenil and others), phenoxyalkane acid derivatives (2,4-D and others), aryl alkane acid derivatives (Fenac (for 2,3,6-trichlorophenyl acetic acid) and others), aniline derivatives (fluchloralin and others), uracil derivatives (lenacil and others), pyridazone derivatives (chloridazon and pyrazon and others), thiourea derivatives (ANTU and others), coumarin derivatives (coumafuryl and others), aryl alkanol derivatives (ancymidol and others), indolyl derivatives (indolylacetic acid and others), dialkane acid derivatives (maleic acid hydrazide and others), chloralkane ether derivatives (octachlorodipropyl ether and others), and sulfoxide derivatives (sulfoxides and others) can be used in accordance with the present invention.

The term “foodstuff” encompasses all food items including, but not limited to, baked goods, including bread, bread dough, cakes, biscuits, pies, rolls and the like; breakfast cereals; candy including chewing gum and chocolate; gelatin desserts; diary products including ice cream, cheese, yogurt, and milk; vegetable oil, beverages including fruit drinks, tea, coffee, beer, wine and soft drinks; shortening including butter, vegetable oil, and margarine; cured meats; non-dairy whiteners; potato chips; whipping agent; artificial whipped cream, processed egg whites; jelly; infant formula; salad dressing including mayonnaise and sandwich spreads.

Suitable adjuvants, diluents and carriers that are useful in preparing the herbicidal, pesticidal and pharmaceutical mixtures of the invention are well known to those skilled in the art.

Liquid carriers that can be employed include water, toluene, xylene, petroleum naphtha, crop oil, acetone, methyl ethyl ketone, cyclohexanone, trichloroethylene, perchloroethylene, ethyl acetate, amyl acetate, butyl acetate, propylene glycol monomethyl ether and diethylene glycol monomethyl ether, methanol, ethanol, isopropanol, amyl alcohol, ethylene glycol, propylene glycol, glycerine, N-methyl-2-pyrrolidinone, and the like. Water is generally the carrier of choice for the dilution of concentrates.

Suitable solid carriers include talc, pyrophyllite clay, silica, attapulgus clay, kieselguhr, chalk, diatomaceous earth, lime, calcium carbonate, bentonite clay, Fuller's earth, cotton seed hulls, wheat flour, soybean flour, pumice, wood flour, walnut shell flour, lignin, and the like.

Other adjuvants commonly utilised in compositions include compatibilising agents, antifoam agents, sequestering agents, neutralising agents and buffers, corrosion inhibitors, dyes, odorants, spreading agents, penetration aids, sticking agents, dispersing agents, thickening agents, freezing point depressants, antimicrobial agents, and the like.

The concentration of the active agents will obviously depend upon the end use and mode of action of the active agent. For example, with respect to herbicidal compositions of this invention is generally from about 0.001 to about 98 percent by weight. Concentrations from about 0.01 to about 90 percent by weight are often employed. In compositions designed to be employed as concentrates, the active agent is generally present in a concentration from about 5 to about 98 weight percent, preferably about 10 to about 90 weight percent. Such compositions are typically diluted with an inert carrier, such as water, before application.

Pesticidal active agent may be used alone; however, usually they are formulated into conventional forms such as dust, granule, microgranule, wettable powder, flowable powder, emulsion, microcapsule, oil, aerosol, etc., using techniques well known in the art. To improve or stabilise the effects of the pesticide, the pesticide is blended with suitable adjuvants and then used as such or after dilution if necessary. Examples of adjuvants include carriers, diluents, spreaders, emulsifying agents, wetting agents, dispersion agents, or fixing agents.

The amount of pharmaceutical active agent that may be combined with the carrier materials to produce a single dosage form will vary depending upon the host treated and the particular mode of administration. For example, a formulation intended for the oral administration of humans may vary from about 5 to about 95% of the total composition. Dosage unit forms will generally contain between from about 1 mg to about 500 mg of active agent.

Having identified a substance for use in the present invention it is activated as defined above. Preferably the substance or a component of the substance is vortexed for a period between 45 and 90 minutes as described below and then agitated for 45 and 90 minutes as described below to produce a fundamental quantum harmonic of between 20 to 50 Hz.

The vortexing and agitation may be by any means capable of forming the desired harmonic as described below. Suitable means include using static mixers (Maa, et al., J. Microencapsulation 13(4): 419-33 (1996)), as well as dynamic mixing means such as agitators, homogenizers, sonicators, and other process equipment known in the art.

In one embodiment, the agitation is performed by blending the dry substance or active agents together as described above with one or more acceptable diluents, carriers or excipients then vortexing and agitating the substance or active agents through a length of pipe or tubing at conditions sufficient to create the desired harmonic, ie. enough turbulence to induce harmonic formation.

Other static devices, such as restriction plates (flow constrictors) and filters, also can be used to create the required harmonic. In a preferred embodiment, non-static mixers are used as the agitation means. As used herein, the term “non-static mixer” refers to a device having elements that freely move within a flowing stream of the fluids to be agitated. Examples of non-static mixers include non-motorised turbines and certain flow indicators, such as a ball indicator. Another example is a flow though mixer head available on a Silverson homogeniser. Non-static mixers advantageously provide more efficient agitation than that induced by turbulent flow alone, and can be less expensive than most dynamic and static mixers. These types of static and non-static mixing means can be used to enhance or replace conventional agitation techniques, such as agitators and static mixers, which may be particularly useful when the process for making the nutrient formulation or composition of the invention is operated continuously at certain production rates. Mixing in a classic static mixer relies on a number of factors, including the rate of fluid flow. Pumps or pressure controls the fluid flow rate and can vary with pump oscillations or changing pressure. The use of a non-static mixer in a continuous process can overcome these oscillations by providing additional steady mixing, resulting in a more consistent emulsion. One of skill in the art can readily optimise these mixing means to achieve the most efficient production of the desired harmonic.

Without wishing to be bound by any theory or hypothesis the applicant believes that by vortexing and agitating the substance or active agent as described herein a vortex in the substance or active agent of the invention produces small amounts of rotons depending on speed and energy of the vortex. Rotons are second generation tachyons formed in oscillating vortex (See, for example, Shatskiy, A A, J. High Energy Phys.: 11 (2001), pp. 064; Pismen, L. Phys. Rev. 2002, pp. 8). This oscillation is fundamental in producing the harmonics which are the basis of the present invention.

In one particularly preferred embodiment the vortex is between 100 mm and 250 mm Radius and has a velocity to impart of between 50 to 100 joules per second.

Calculation of the conditions to produce the specific harmonic is as follows:
˜K^{d}+Gₜ^{np}+Σ^{Eg}M=0
where K^{d}=Thermal Density of Fluid
Gₜ^{np} ((T+F+R)\hat{ }-Pi
T=TEMPERATURE
Σ^{g}=HARMONIC MEAN OF FLUID
F=DESIRED HARMONIC FLUID
M=Mass of Fluid
R=Energy imparted to fluid

The harmonic may be measured by a protek multifunction counter 9100 or similar frequency meter. This is done by immersing a probe into the liquid formulation after agitation has occurred. The reading is then taken of the fundamental harmonic of the agitated liquid.

In a preferred method the substance or active agent described above is vortexed at a low velocity to form a vortex in one direction of between 30-120 rpm at which point the direction of vortex is reversed until the vortex reaches a velocity of between 30-120 rpm at which point the direction of the vortex is reversed again and so repeated until a period of 45 minutes to 90 minutes is reached.

While it is possible to use any vortex machine to produce the appropriate vortex it is preferable that the system uses the kinetic energy of isotropic fluids of a range between 40,000 and 80,000 kJ.

Once the appropriate vortex has been formed in the substance or active agent it is then agitated at a rate of between 50,000-65,000 Kj/mole at an angle of 10-90 degrees at a frequency between 0.1-100 cycles per second. During this step the solution is energised. This stage lasts between 45 to 90 minutes.

The substance or active agent may then succussed in the agitator at a rate of 50000-65000 kj/mole at a angle 10-90 Degrees at a frequency between 0.1-100 cycles per second. During this step the solution is energized. This stage lasts between 40 to 80 minutes. This solution is either further diluted as in step 1 and returned to step 2 or packaged.

The final agitated substance or active agent can be administered to a subject either as solution, as an ointment or paste, as tablets, or in the form of pellets or globules of a carrier, such as lactose. Alternatively, the substance can be manufactured into foodstuff, pharmacuetical preparations or other such material. It is also possible to triturate the substance or active agent with a solid carrier. Tablets or capsules may be of suitable size which are convenient for swallowing, for example about 0.2 g to about 1 g. The final substance may also be a liquid or a powder and may be added to other substances which may not be produced by this process to make a final medicine or substance.

The substance or active agent can then either containerised or potentised further as follows:

1 ml or 1 g of substance or active agent is mixed with 9 ml of diluent to produce 10 ml of 1× attenuation. This is then vortexed and rotated then agitated as described below where it is succussed. A further dilution of the processed substance or active agent can then be made as necessary by taking 1 ml of 1× attenuation which is succussed with 9 mls of diluent to produce 10 ml of 2× attenuation and so on. This may be repeated until the desired potency is achieved.

In one embodiment rather than blending the substance or active agent then vortexing and agitating the entire substance, formulation or composition as described above it is possible to merely vortex one or more of the agents separately then blend these agents together. For example, 1 gram of substance eg medicament, trace element, mineral, plant or animal material may be added to a volume of liquid of 15,000 to 20,000 L and then vortexed and succussed resulting in a biomorphogenic medicine.

The term “biomorphogenic” as used herein refers to the enhancement of the electrical potential of a substance by the creation of fundamental harmonic profiles as described throughout the specification.

With respect to pharmaceutical substances or active agents of the present invention these may be administered orally, topically, parenterally, or by inhalation spray in dosage unit formulations containing non-toxic pharmaceutically acceptable carriers, adjuvants and vehicles. The term parenteral as used herein includes subcutaneous injections, intravenous, or intramuscular.

A pharmaceutical substance or active agent of the invention may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from sweetening agents, flavouring agents, colouring agents and preserving agents in order to provide pharmaceutically elegant and palatable preparations. Tablets contain the active agent in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastointestinal tract and thereby provide a sustained action over a longer period. For example, a time delay material such as glyceryl monostearate or glyceryl distearate may be employed. They may also be coated by the techniques described in the U.S. Pat. No. 4,256,108, U.S. Pat. No. 4,166,452 and U.S. Pat. No. 4,265,874, to form osmotic therapeutic tablets for controlled release.

Formulations for oral use may also be presented as hard gelatin capsules where in the active agent is agitate with an inert solid diluent, for example calcium carbonate, calcium phosphate or kaolin, or as soft gelatin capsules wherein the active agent is agitate with water or an oil medium, for example peanut oil, liquid paraffin or olive oil. Aqueous suspensions contain the active materials in admixture with excipients suitable for the manufacture of aqueous suspensions. Such excipients are suspending agents, for example sodium carboxymethylcellulose, methylcellulose, hydroxy-propylmethylcellulose, sodium alginate polyvinyl-pyrrolidone, gum tragacanth and gum acacia; dispersing or wetting agents may be a naturally occurring phosphatide, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such a polyoxyethylene with partial esters derived from fatty acids and hexitol anhydrides, for example polyoxyethylene sorbitan monooleate. The aqueous suspensions may also contain one or more preservatives, for example ethyl, or n-propyl, p-hydroxybenzoate, one or more colouring agents, one or more flavouring agents, and one or more sweetening agents, such as sucrose or saccharin.

Oily suspensions may be formulated by suspending the active agent in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavouring agents may be added to provide a palatable oral preparation. These compositions may be preserved by the addition of an anti-oxidant such as ascorbic acid.

Dispersible powders and granules suitable for preparation of an aqueous suspension by the addition of water provide the active agent in admixture with a dispersing or wetting agent, suspending agent and one or more preservatives. Suitable dispersing or wetting agents and suspending agents are exemplified, for example sweetening, flavouring and colouring agents may also be present.

The substance or active agent of the invention may also be in the form of oil-in-water emulsions. The oily phase may be a vegetable oil, for example olive oil or arachis oil, or a mineral oil, for example liquid paraffin or mixtures of these. Suitable emulsifying agents may be naturally occurring gums, for example gum acacia or gum tragacanth, naturally occurring phosphatides, for example soya bean, lecithin, and esters or partial esters derived from fatty acids and hexitol anhydrides, for example sorbitan monooleate and condensation products of the said partial esters with ethylene oxide, for example polyoxyethylene sorbitan monooleate. The emulsions may also contain sweetening and flavouring agents.

Syrups and elixirs may be formulated with sweetening agents, for example glycerol, propylene glycol, sorbitol or sucrose or lactose. Such formulations may also contain a demulcent, a preservative and flavouring and colouring agents. The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be in a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

Aerosols of liquid particles comprising the pharmaceutical substance or active agent of the invention may be produced by any suitable means, such as with a nebuliser. See, eg., U.S. Pat. No. 4,501,729. Nebulisers are commercially available devices which transform solutions or suspensions of the active agent into a therapeutic aerosol mist either by means of acceleration of a compressed gas, typically air or oxygen, through a narrow venturi orifice or by means of ultrasonic agitation. Suitable formulations for use in nebulisers consist of the active agent in a liquid carrier, the active agent comprising up to 40% w/w, but preferably less than 20% w/w, of the formulation. The carrier is typically water or a dilute aqueous alcoholic solution, preferably made isotonic with body fluids by the addition of, for example, sodium chloride. Optional additives include preservatives if the formulation is not prepared sterile, for example, methyl hydroxybenzoate, antioxidants, flavouring agents, volatile oils, buffering agents and surfactants.

The aerosols of solid particles comprising the active agent may likewise be produced with any solid particulate medicament aerosol generator. Aerosol generators for administering solid particulate medicaments to a subject produce particles which are respirable, as explained above, and generate a volume of aerosol containing a predetermined metered dose of a medicament at a rate suitable for human administration. One illustrative type of solid particulate aerosol generator is an insufflator. Suitable formulations for administration by insufflation include finely comminuted powders which may be delivered by means of an insufflator or taken into the nasal cavity in the manner of a snuff. In the insufflator, the powder, eg., a metered dose thereof effective to carry out the treatments described herein, is contained in capsules or cartridges, typically made of gelatin or plastic, which are either pierced or opened in situ and the powder delivered by air drawn through the device upon inhalation or by means of a manually-operated pump. The powder employed in the insufflator consists either solely of the active agent or of a powder blend comprising the active agent, a suitable powder diluent, such as lactose, and an optional surfactant. The active agent typically comprises from 0.1 to 100 w/w of the formulation.

A second type of illustrative aerosol generator comprises a metered dose inhaler. Metered dose inhalers are pressurised aerosol dispensers, typically containing a suspension or solution formulation of the active agent in a liquefied propellant. During use these devices discharge the formulation through a valve adapted to deliver a metered volume, typically from 10 to 150 μl, to produce a fine particle spray containing the active agent. Suitable propellants include certain chlorofluorocarbon compounds, for example, dichlorodifluoromethane, trichlorofluoromethane, dichlorotetrafluoroethane and mixtures thereof. The formulation may additionally contain one or more co-solvents, for example, ethanol, surfactants, such as oleic acid or sorbitan trioleate, antioxidants and suitable flavouring agents.

The aerosol, whether formed from solid or liquid particles, may be produced by the aerosol generator at a rate of from about 10 to 150 litres per minute, more preferably from about 30 to 150 litres per minute, and most preferably about 60 litres per minute. Aerosols containing greater amounts of medicament may be administered more rapidly.

In one particular embodiment the substance or active agent of the present invention further comprises boron, which appears to help maintain calcium balance, keeping bones healthy and preventing osteoporosis. Preferably, adequate levels of boron (˜3-5 mg) in the diet to maintain healthy bones is required. Zinc may also be included as it has been shown to reduce joint swelling and other symptoms in rheumatoid arthritis.

In a further preferred embodiment the substance or active agent of the present invention further comprises calcium. Calcium supplementation given at a 400 mg dose twice a day twice daily had been shown to avert bone loss and stabilized bone density in the spine, femoral neck, and radial shaft in women relatively soon after menopause.

In one embodiment the present invention provides a composition for use in the prevention and/or treatment of a medical disorder mediated in whole or part by mineral deficiency and free radicals, comprising:
at least one vitamin;
at least one trace element; and
one homoeopathic and/or biomorphogenic ingredient.

Preferably, the vitamin is vitamin C, the trace elements comprise one or more of magnesium, boron, zinc and sodium. Preferably, the calcium is in the form of calcium citrate or calcium carbonate. The preferred composition also comprises ascorbic acid, sodium bicarbonate, magnesium aspartate or magnesium orotate, seleno-methionine, boron and either zinc oxide or zinc aspartate.

In one preferred embodiment the invention provides a pharmaceutical substance or composition comprising:

| | |
|---|---|
| Ascorbic acid | equivalent 30 to 250 mg/g |
| Calcium | equivalent 80 to 100 mg/g |
| Magnesium | equivalent 2 to 2.5 mg/g |
| Zinc (picolinate) | equivalent 3 to 20 mg/g |
| Selenomethionine | equivalent 0.002 to 0.0090 mg/g |
| Na Bicarbonate | equivalent 180 to 205 mg/g |
| Boron | equivalent 0.001 to 0.005 |

Without wishing to be bound by any theory or hypothesis the applicant believes that the method of the present invention further preferably results in the scavenging of free radicals by the Phase I cytochrome P₄₅₀ system of the liver and the production of water soluble metabolites of toxic xenobiotics via the Phase I cytochrome P₄₅₀ of the liver. The Phase I cytochrome P₄₅₀ enzymes are believed to be benefited by the presence of vitamin C, selenomethionine and zinc.

During Phase II cytochrome P₄₅₀ of the liver is further supported by the nutrient formulation by provision of mineral replacement, thereby supporting eliminatory organs—including the kidneys, and the cardiovascular system, including the heart and circulatory system and also to correct mineral deficiencies.

In one especially preferred embodiment of the invention the method of the present invention may be used to treat and/or prevent any one or a combination of the following conditions arthritis, osteoporosis, tendonitis, fibromyalgia, trauma injury to soft tissues such as ligaments and tendons or other to relieve symptoms caused by mineral deficiency or assist regulation of immune function in disorders caused by free radical activity. This formulation shall be used to correct metabolic pathways caused by enzyme deficiency due to vitamin and mineral deficiencies. The purpose of administering the dietary composition to patients is to stimulate certain enzymes of the body which when sufficiently active are capable of clearing from the body numerous accumulated undesirable non-end product metabolites and toxins. Sources of such non-end product metabolites and toxins may be environmental, such as exposure to environmental xenobiotic substances—ie. heavy metals, pesticides, herbicides, fungicides, altered DNA fractions, poisons, certain drugs and pharmaceuticals, as well as excessive levels of other non-end product metabolites which are formed in biochemical reactions in the body during states of altered metabolism. The human body's ability to enzymatically process undesirable metabolites and toxins is demonstrably enhanced as a result of treatment in accordance with the present invention.

It will be understood, however, that the specific dose level for any particular subject will depend upon a variety of factors including the activity of the specific compound employed, the age, body weight, general health, sex, diet time of administration, route of administration, rate of excretion, drug combination and the severity of the particular airway disease undergoing therapy.

In one embodiment, the substance comprises a liquid consisting of dry agents blended together. One particularly preferred nutrient formulation comprises ascorbic acid (about equivalent 350 to 600 mg/g, calcium citrate (about equivalent 60 to 80 mg/g, magnesium aspartate (about equivalent 0.9 to 1.6 mg/g, zinc picolinate (about equivalent 1 to 2 mg/g, selenomethionine (about equivalent 0.005 to 0.01 mg/g, Na bicarbonate (about equivalent 130 to 140 mg/g boron from a homoeopathic or morphogenic source between 1× and 20×, and probiotic bacteria between 1 to 10¹¹ cfu per gm blended together with between 400 ml to 1000 ml water and 2% of a suitable “non toxic surfactant”. The term “non toxic surfactant” may include lecithin or glycerol, potassium sorbate and ethanol. The method of blending of the dry agents, water and surfactant is not essential and any standard techniques used in the art may be employed.

The preferred formulation or composition may also include a nutritionally acceptable soluble magnesium salt, for example in the form of magnesium aspartate or orotate. Other additives include soluble calcium salt, ascorbic acid derivative, for example calcium citrate, orotate or carbonate, sodium, potassium, magnesium aspartate or orotate, zinc ascorbate or picolinate or aspartate or oxide; ascorbic acid, or as zinc amino acid chelate, boron, selenomethionine as well as pharmaceutically acceptable buffering salt such as, for example, sodium bicarbonate.

Co-pending application PCT/AU03/00103 (incorporated herein by reference) describes a specific airway disorder formulation. Consequently, the present application explicitly excludes such a formulation by proviso.

Throughout the specification, unless the context requires otherwise, the word “comprise” or variations such as “comprises” or “comprising”, will be understood to imply the inclusion of a stated integer or group of integers but not the exclusion of any other integer or group of integers.

The invention will now be further described by way of reference only to the following non-limiting examples. It should be understood, however, that the examples following are illustrative only, and should not be taken in any way as a restriction on the generality of the invention described above. In particular, while the invention is described in detail in relation to a specific asthma formulation, it will be clearly understood that the findings herein are not limited to this formulation. For example, other formulations for other airway disease may be produced using the techniques herein described as long as they comprise the harmonic disclosed.

### EXAMPLE 1

### Activation of Water

The frequency of tap water before energizing was 0. The Optical absorption of the water before excitation was 1.1 as measured by Gallenkamp calorimeter in white light. The main vortex operated at 18 rpm with a reversal at 6 seconds and with a gap of 4 second. The water was processed in the vortex processor for one hour. It reached initial primary frequency of 9.75 Hz after about 15 minutes. After 60 minutes the first process was stopped and water transferred to the second (succussion) stage. Its frequency before this stage was 249 Hz; during this stage the frequency was 9.8 Hz. The succussion rate was between 1 and 8 succussions per second. This process lasted 60 minutes. The final frequency was 31.8 Hz. The Optical absorption of the water after excitation was 0.4 (measured by colourimeter).

### EXAMPLE 2

### Activation of Milk

The frequency before energizing was 6.6 Hz. The main vortex operated at 18 rpm with a reversal at 5 seconds and with a gap of 5 seconds. The milk was processed in the vortex processor for one hour. It reached initial primary frequency of 9.81 Hz after about 12 minutes. After 60 minutes the first process was stopped and the milk transferred to the second (succussion) stage. Its frequency before this stage was 227 Hz. During this stage the frequency was 9.6 Hz. The succussion rate was between 1 and 8 succussions per second. This process lasted 60 minutes. The Final frequency was 31.08 Hz. As shown in Table 1 milk showed pronounced biological activity after excitation by increased lactobacillus growth during later culturing in a laboratory setting.

TABLE 1

| | Bacterial | Bacterial | Percentage |
|---|---|---|---|
| | Growth Before | Growth After | Increase after |
| | Cfu/ml | Cfu/ml | Excitation of milk |
| Lactobacillus | 140000000 | 850000000 | 607.1428571 |
| acidophilus | | | |
| Lactobacillus | 300000000 | 860000000 | 286.6666667 |
| plantarum | | | |
| Lactobacillus | 3200000 | 1900000000 | 59375 |
| brevis | | | |
| Lactobacillus | 4200000 | 980000000 | 23333.33333 |
| delbruccei | | | |
| Lactobacillus | 400000 | 5000000000 | 1250000 |
| salivarus | | | |
| Bifido | 400000 | 110000000 | 27500 |
| Bacterium | | | |

### EXAMPLE 3

### Asthma Formulation Preparation

The applicant produced an asthma medicament as follows:

| | |
|---|---|
| Ascorbic acid | equivalent 250-350 mg/g |
| Calcium citrate | equivalent 55 to 62 mg/g |
| Magnesium aspartate | equivalent 2 to 2.5 mg/g |
| Zinc oxide | equivalent 9.64 to 21 mg/g |
| Selenomethionine | equivalent 0.01 to 0.10 mg/g |
| Na Bicarbonate | equivalent 140 mg/g to 180 mg/g |
| Boron | equivalent 0.00000001 to 0.05 mg/g |
| Probiotic Bacteria | between 1 to 10¹¹ cfu per gm. |

These ingredient were blended together. Daily dosages could range from between 0.125 mg for infants up to about 6 grams for adults. In order to produce a liquid formulation the appropriate dosage amounts of the formulation was mixed with between 400 to 1000 ml of water and 2% surfactant was added.

The formulation was then vortexed for 45-90 minutes at 30-120 rpm as described above to produce the fundamental quantum harmonic of between 20 to 50 Hz as measured by protek multifunction counter 9100 frequency meter.

Table 2 shows a series of frequency measurements taken by protek multifunction counter 9100 frequency meter

TABLE 2
EXAMPLES OF FREQUENCIES OF DIFFERENT FLUID MEDIUMS

| | | | End | | | | End | |
|---|---|---|---|---|---|---|---|---|
| | Initial | Vortex | Vortex | Vortex | Time | Succussion | Succussion | Time |
| Material | Frequency | Frequency | Frequency | Speed | Vortex | Frequency | Frequency | Succussion |
| Water | 0 | 9.75 | 249 | 18 | 60 | 9.8 | 31.8 | 60 |
| Milk | 6.6 | 9.81 | 227 | 18 | 60 | 9.6 | 31.01 | 60 |
| Nutrient | 5.9 | 9.819 | 239 | 18 | 60 | 9.85 | 31.65 | 55 |

of liquids prior to agitation and after agitation.

The experimental data shown in Table 3 indicates that energy was imparted into the liquid medium during the vortexing and agitating process. This is further proven by the measurement of frequencies of the liquid medium before and after processing which show improvements of >100%. All frequencies were measured by protek multifunction counter 9100 frequency meter method.

Bioresonance testing was completed on the fluid mediums of H₂O, milk and liquid nutrient formulation. These were tested by the Bioresonance Method of Schimmel (Schimmel, H 1986, Bioenergetic Regulatory Techniques VEGA Gieshaber GmbH & Co Am Hohenstein 113 PO 1142D 7-622 Scitach Germany). Increases in resonance show improvements of between 20 and 40%. The optical density was measured by Englehart colorimeter and showed improvements of >75%.

The frequencies of the post agitation frequencies remained constant at a range of between 20 and 50 Hz and revealed that the fundamental harmonic of the agitated materials H₂O, milk and nutrient formulation to be maintained and therefore a stable biomorphogenic end product attained.

Once produced the formulation was then ready to be administered to patients as a medicine in order to stimulate certain enzymes of the body which when sufficiently active are capable of clearing from the body numerous accumulated undesirable non-end product metabolites and toxins

TABLE 3
EXAMPLES OF BIORESONANCE AND OPTICAL CHARACTERISTICS

| | | Increase | Pre-Optical | Post-Optical |
|---|---|---|---|---|
| Bioresonance % | Bioresonance % | Bioresonance % | Characterisitic | Characterisitic |
| 45 | 85 | 40 | 1.1 | 0.4 |
| 80 | 100 | 20 | na | na |
| 80 | 100 | 20 | 1.9 | 1.1 |

### EXAMPLE 4

### Asthma Medicament in Water

Between 30-75 g of powdered asthma medicine as described in example 3 was added to a volume of water between 500 ml and 20,000 ml. The frequency before energizing was 5.9. The optical absorption of the water before excitation was 1.9 as measured by Gallenkamp colorimeter in white light. The main vortex operated at 18 rpm with a reversal at 6 seconds and with a gap of 4 second. The solution was processed in the vortex part for one hour. It reached initial primary frequency of 9.81 Hz after approximately 15 minutes. After 60 minutes the first process was stopped and solution transferred to the second (succussion) Stage. Its frequency before this stage was 239 Hz. During this stage the frequency was 9.85 Hz. The succussion rate was between 1 and 8 succussions per second. This process lasted 60 minutes. The Final frequency was 31.65 Hz, The Optical absorption of the solution after excitation was 1.1.

### EXAMPLE 5

### Dilution of Asthma Medicament in Water

10 mls of solution obtained from the end of processing in Example 4 was mixed with 20 litre of water. The frequency before energizing was 8.2. The Optical absorption of the water before excitation was 1.9 as measured by Gallenkamp colorimeter in white light. The main vortex operated at 18 rpm with a reversal at 6 seconds and with a gap of 4 second. The solution was processed in the vortex part for one hour. It reached initial primary frequency of 9.79 Hz after 15 minutes. After 60 minutes the first process was stopped and solution containing the medicine transferred to the second (succussion) stage. Its frequency before this stage was 241 Hz. During this stage the frequency was 9.81 Hz. The succussion rate was between 1 and 8 succussions per second. This process lasted 60 minutes. The Final frequency was 31.01 Hz. The Optical absorption of the solution after excitation was 1.1 as measured by Gallenkamp colorimeter in white light.

### EXAMPLE 6

### Asthma Medicament in Water

10 mls of solution obtained from the end of processing Example 5 was mixed with 20 litre of water. The frequency before energizing was 8.2. The Optical absorption of the water before excitation was 1.9 as measured by Gallenkamp calorimeter in white light. The main vortex operated at 18 rpm with a reversal at 6 seconds and with a gap of 4 second. The solution was processed in the vortex part for one hour. It reached initial primary frequency of 9.81 Hz after 15 minutes. After 60 minutes the first process was stopped and solution transferred to the second (succussion) stage. Its frequency before this stage was 239 Hz. During this stage the frequency was 9.8 Hz. The succussion rate was between 1 and 8 succussions per second. This process lasted 60 minutes. The Final frequency was 31.65 Hz. The Optical absorption of the solution after excitation was 1.1.

### EXAMPLE 7

### Antioxidant Medicament in Water

An antioxidant medicament as shown in Table 4, was produced by the method of Example 1.

TABLE 4
Dosage of antioxidant subcutaneous and or intravenous or
intramuscular medicine

| | Minimum range | Maximum range |
|---|---|---|
| | mg/ml | mg/ml |
| Ascorbic acid | 0.1 | 2 |
| Calcium | 0.1 | 2 |
| Magnesium | 0.001 | 1 |
| Zinc picolinate | 0.001 | 2 |
| seleno-methionine | 0.00001 | 0.1 |
| Na bicarbonate | 0.1 | 2 |
| Boron | 0.00001 | 2 |
| Probiotics measured in cfu/ml | | |
| Lactobacillius acidophilus | 1 × 10¹ | 1 × 10¹¹ |
| Lactobacillius brevis | 1 × 10¹ | 1 × 10¹¹ |
| Lactobacillius casei | 1 × 10¹ | 1 × 10¹¹ |
| Lactobacillius delbruceii | 1 × 10¹ | 1 × 10¹¹ |
| Lactobacillius rhamnosus | 1 × 10¹ | 1 × 10¹¹ |
| Lactobacillius rhamnosus | 1 × 10¹ | 1 × 10¹¹ |
| Lactobacillius plantarum | 1 × 10¹ | 1 × 10¹¹ |
| Lactobacillius salivarus | 1 × 10¹ | 1 × 10¹¹ |
| BiffidoBacterium bifidum | 1 × 10¹ | 1 × 10¹¹ |

The frequency before energizing was 8.3. The Optical absorption of the water before excitation was 1.9 as measured by Gallenkamp colorimeter in white light. The main vortex operated at 18.5 rpm with a reversal at 6 seconds and with a gap of 4 second. The solution was processed in the vortex part for one hour. It reached initial primary frequency of 9.81 Hz after about 15 minutes. After 60 minutes the first process was stopped and solution transferred to the second (succussion) stage. Its frequency before this stage was 246 Hz During this stage the frequency was 9.75 Hz. The succussion rate was between 1 and 8 succussions per second. This process lasted 60 minutes. The Final frequency was 31.09 Hz. The Optical absorption of the solution after excitation was 1.1 as measured by Gallenkamp calorimeter in white light.

### EXAMPLE 8

### Antioxidant Medicament—IV/SC Injection

Between 30-75 g of powdered antioxidant medicine as described in Example 7 was added to a volume of physiological saline of between 500 ml and 20,000 ml. The frequency before energizing was 4.67. The Optical absorption of the water before excitation was 1.9. The main vortex operated at 18.5 rpm with a reversal at 6 seconds and with a gap of 4 second. The solution was processed in the vortex part for one hour. It reached initial primary frequency of 9.81 Hz after about 15 minutes. After 60 minutes the first process was stopped and solution transferred to the second (succussion) Stage. Its frequency before this stage was 251 Hz. During this stage the frequency was 9.81 Hz The succussion rate was between 1 and 8 succussions per second. This process lasted 60 minutes. The Final frequency was 31.09 Hz. The Optical absorption of the solution after excitation was 1.1 as measured by Gallenkamp colorimeter in white light.

### EXAMPLE 9

### Clinical Trial of Antioxidant Medicine on Guinea Pigs

A sample of the antioxidant medicament described in Example 8 was made into a subcutaneous/intravenous solution by sterilising it in a standard hospital autoclave. A measured volume of sterilised solution of between 0.1 and 1 ml was then injected into a guinea pig. No adverse skin eruption at the site of injection or adverse side effect was noted following injections over several days.

Previous work by Linus Pauling institute has also demonstrated safety of high dose ascorbic acid in guinea pigs. These studies were conducted to investigate whether ascorbic acid protected guinea pigs from aflatoxin B1 (AFB1) toxicity. Young guinea pigs, fed either 0 (AA) or 25 mg (25 AA) or gavaged 300 mg ascorbic acid (300 AA) per day for 21 days, were gavaged with the LD50 dose of AFB1 on the 22nd day. Seven out of 10 animals in the AA group died within 72 hr of AFB1 administration. The livers of the animals showed regional massive necrosis and multilobular degeneration. There was no mortality in the 25 AA group. Their livers, however, showed changes similar to those seen in AA group. Serum alanine amino transferase (ALAT) and aspartate amino transferase (ASAT) levels were elevated. There was neither mortality nor pathological changes in livers in the 300 AA group. Their ALAT and ASAT levels were unaffected. In vitro production of AFM1 by liver microsomes tended to be higher than that in the other two groups. Three animals saved from the 300 AA group and continued with their supplementation were administered a second, intraperitoneal (ip) LD50 dose of AFB1 1 month after the first AFB1 dose. One animal died. Livers of the animals showed centrilobular degeneration and moderate necrosis in scattered hepatocytes. Liver microsomal cytochrome P450 and cytosolic glutathione S-transferase (GST) levels and AFM1 production were drastically reduced. ALAT and ASAT activities were raised. The results indicated that intake of 300 mg of ascorbic acid almost protected the animals from acute toxicity of AFB1 when given by gavage, but not when administered as a second dose ip.

### EXAMPLE 10

### Clinical Trial of Antioxidant Medicament in a Herd of Dairy Goats

A sample of the antioxidant medicament described in Example 8 was made into a subcutaneous/intravenous solution by sterilising it in standard hospital autoclave. A measured volume of sterilised solution of between and 1 ml and 2 mls was then injected subcutaneously into 100 goats. No adverse skin eruption at the site of injection or adverse side effect was noted in the goats following injections over several days and weeks.

Prior to the subcutaneous injections of the goats, milk obtained from the goats was cultured on agar plates. Upon microbial examination at a hospital laboratory, Lactococcus lactis and Enterococcus durans were observed. These organisms had grown to a count of over 1 million organisms per ml in the milk and were causing exotoxins and enterotoxins to be released. This was resulting in gut stasis and death in 12 goats. Following 2 subcutaneous injection of the antioxidant medicament there were no more reported deaths or signs of illness. Moreover, the bacterial count for Lactococcus lactis and Enterococcus durans had dropped from over 1×10⁶ to less than 3,400 for each species.

### EXAMPLE 11

### Asthma Medicament—IV/SC Injection

Between 30-75 g of powdered asthma medicament as described in Example 3 was added to a volume of water between 500 ml and 20,000 ml. The frequency before energizing was 4.34. The optical absorption of the water before excitation was 1.9 as measured by Gallenkamp calorimeter in white light. The main vortex operated at 18 rpm with a reversal at 6 seconds and with a gap of 4 second. The solution was processed in the vortex part for one hour. It reached initial primary frequency of 9.75 Hz after 15 minutes. After 60 minutes the first process was stopped and solution transferred to the second (succussion) Stage. The frequency before this stage was 239 Hz. During this stage the frequency was 9.8 Hz. The succussion rate was between 1 and 8 succussions per second. This process lasted 60 minutes. The Final frequency was 31.08 Hz. The Optical absorption of the solution after excitation was 1.1 as measured by Gallenkamp calorimeter in white light.

### EXAMPLE 12

### Clinical Trial of Asthma Medicament in Guinea Pig

A sample of the asthma medicament described in Example 11 was made into a subcutaneous/intravenous solution by sterilising it in standard hospital autoclave. A measured volume of sterilised solution of between 0.1 and 1 ml was then injected into a guinea pig. No adverse skin eruption at the site of injection or adverse side effect was noted following injections over several days.

### EXAMPLE 13

### Clinical Trial of Asthma Medicament in Herd of Dairy Goats

A sample of the asthma medicament described in Example 11 was made into a subcutaneous/intravenous solution by sterilising it in standard hospital autoclave. A measured volume of sterilised solution of between and 1 ml and 2 mls was then injected subcutaneously into 100 goats. No adverse skin eruption at the site of injection or adverse side effect was noted in the goats following injections over several days and weeks.

### EXAMPLE 14

### Activation of Ginseng

A preparation of the herb Ginseng was made by soaking some ginseng root in vinegar overnight. This was then pureed the next day, filtered and the resulting filtrate added to a volume of water between 500 ml and 20,000 ml of water. The frequency before energizing was 3.65. The main vortex operated at 18.5 rpm with a reversal at 6 seconds and with a gap of 4 second. The solution was processed in the vortex part for one hour. It reached initial primary frequency of 9.6 Hz after about 15 minutes. After 60 minutes the first process was stopped and solution transferred to the second (succussion) stage. Its frequency before this stage was 255 Hz. During this stage the frequency was 9.8 Hz. The succussion rate was between 1 and 8 succussions per second. This process lasted 60 minutes. The Final frequency was 31.08 Hz.

### EXAMPLE 14

### Preparation of Snail Repellent

A snail repellent was made by taking a mature snail and soaking it in vinegar overnight. The snail was then pureed the next day and added to a volume of water between 500 ml and 20,000 ml. The frequency before energizing was 0.18. The optical absorption of the water before excitation was 2 as measured by Gallenkamp colorimeter in white light. The main vortex operated at 18.5 rpm with a reversal at 6 seconds and with a gap of 4 second. The solution was processed in the vortex part for one hour. It reached initial primary frequency of 9.81 Hz after approximately 15 minutes. After 60 minutes the first process was stopped and solution transferred to the second (succussion) stage. Its frequency before this stage was 255 Hz During this stage the frequency was 9.75 Hz. The succussion rate was between 1 and 8 succussions per second. This process lasted 60 minutes. The Final frequency was 31.65 Hz. The Optical absorption of the solution after excitation was 1.2 as measured by Gallenkamp colorimeter in white light.

When the repellent had completed the frequency process (Vortex and succussion) it was sprayed on a test snail. Before spraying the snail had a frequency of 5.6, after 5 minutes this had dropped to 3.4. Within 45 minutes this had dropped to 1.6 and within 1 hour the snail was dead.

This was similarly observed on 100 snails in a domestic garden. Those snails not directly sprayed left the vicinity of spraying within a 24 hour period.

### EXAMPLE 15

### Preparation of Moth Repellent

A preparation of a moth was made by taking a mature moth and soaking it in vinegar overnight. The moth was then pureed the next day and added to a volume of water between 500 ml and 20,000 ml of water. The frequency before energizing was 0.36. The main vortex operated at 18.6 rpm with a reversal at 6 seconds and with a gap of 4 second. The solution was processed in the vortex part for one hour. It reached initial primary frequency of 9.81 Hz after approximately 15 minutes. After 60 minutes the first process was stopped and solution transferred to the second (succussion) stage. Its frequency before this stage was 259 Hz. During this stage the frequency was 9.75 Hz. The succussion rate was between 1 and 8 succussions per second. This process lasted 60 minutes. The Final frequency was 31.05 Hz.

### EXAMPLE 16

### Preparation of Fly Repellent

A preparation of a fly repellent was made by taking a mature fly and soaking it in vinegar overnight. The fly was then pureed the next day and added to a volume of water between 500 ml and 20,000 ml of water the next day The frequency before energizing was 0.36. The main vortex operated at 18.6 rpm with a reversal at 6 seconds and with a gap of 4 second. The solution was processed in the vortex part for one hour. It reached initial primary frequency of 9.81 Hz after about 15 minutes. After 60 minutes the first process was stopped and solution transferred to the second (succussion) Stage. Its frequency before this stage was 259 Hz During this stage the frequency was 9.75 Hz The succussion rate is 1 to 8 succussions per second. This process lasted 60 minutes. The Final frequency was 31.05 Hz.

### EXAMPLE 17

### Preparation of Herbicide

A preparation of the “weed” oxalis was made by soaking some oxalis in vinegar overnight. This was then pureed the next day, filtered and the resulting filtrate added to a volume of water between 500 ml and 20,000 ml of water. The frequency before energizing was 1.4. The main vortex operated at 18.6 rpm with a reversal at 6 seconds and with a gap of 4 second. The solution was processed in the vortex part for one hour. It reached initial primary frequency of 9.67 Hz after approximately 15 minutes. After 60 minutes the first process was stopped and solution transferred to the second (succussion) stage. Its frequency before this stage was 259 Hz. During this stage the frequency was 9.68 Hz. The succussion rate was 1 to 8 succussions per second. This process lasted 60 minutes. The Final frequency was 31.07 Hz.

### EXAMPLE 18

### Preparation of Organic Biodynamic Fertiliser

A sample of between 1 and 10 g of Biodynamic Preparation “500”™ was placed with a volume of water between 500 ml and 20,000 mls. The frequency before energizing was 5.8. The main vortex operated at 18.6 rpm with a reversal at 6 seconds and with a gap, of 4 second. The solution was processed in the vortex part for one hour. It reached initial primary frequency of 9.8 Hz after about 15 minutes. After 60 minutes the first process was stopped and water transferred to the second (succussion) stage. Its frequency before this stage was 261 Hz. During this stage the frequency was 9.68 Hz. The succussion rate was 1-8 succussions per second. This process lasted 60 minutes. The Final frequency was 31.08 Hz.

### EXAMPLE 19

### Preparation of Liquid Nutrient Formulation

A liquid nutrient formulation was prepared comprising at between 200 mg/g to 600 mg/g equivalent ascorbic acid; between 50 mg/g to 200 mg/g equivalent calcium citrate carbonate or orotate; between 1.5 mg/g to 20 mg/g equivalent magnesium aspartate or magnesium sulphate or orotate; between 5 mg/g to 30 mg/g equivalent zinc oxide or equivalent zinc picolinate 0.1 mg/g to 5 mg/g; between 0.001 mg/g to 0.1 mg/g equivalent seleno-methionine; equivalent sodium bicarbonate 100 to 300 mg/g equivalent Na content and equivalent boron 0.00000001 mg/g to 2 mg/g. Between 1 cfu and 1×10¹¹ cfu per ml of probiotic bacteria was also added. The probiotic bacteria were Lactobacillus acidophilus; Lactobacillus brevis; Lactobacillus casei; Lactobacillus delbruceii; Lactobacillus rhamnosus; Lactobacillus plantarum; Lactobacillus salivarus and BifidoBacterium bifidum.

In this form the preferred dosage includes a range of between 1× and 1:1000 ratio with dilution of liquid (water or ethanol). The latter being 1:1M homoeopathic following equivalent dilution of powder preparation—i.e. between 1:1 ration and preparation in which dilutions are succussed as followed: 2 ml of tincture is succussed with 8 ml of diluent to produce 10 ml of 2× attenuation, 1 ml of 2× attenuation is then succussed with 9 mls of diluent to produce 10 ml of 3× attenuation and so on. This is repeated until the desired potency is acquired. Suspension in alcohol is the specified menstruum for the final decimal or centesimal attenuation when intended for medical purposes. The amount of alcohol will vary from between 2-60% depending on the desired potency.

### EXAMPLE 20

### Preparation of Powdered Nutrient Formulation

A powdered formulation of the liquid nutrient formulation may be obtained either by blending the ingredients shown without water or lyophilising the liquid nutrient formulation after vortexing and succussion process.

The liquid or powdered nutrient formulation is designed to utilise ingredients which have low allergenic potential or no known tendency to cause allergies, have no artificial chemical residues present on analysis conducted by presently scientifically accepted analytical methods, and contain nutrients and substances which cause increased activity of hepatic enzymes. When used as a total dietary replacement during times of acute dehydration or diarrhoea, the nutrient formulation of the present invention is also designed to provide substantially all nutrients and vitamins required by the human body, and thus to provide a substantially balanced diet.

### EXAMPLE 21

### Preparation of Probiotic Bacteria

The protocol for culturing probiotic bacteria is as follows:

Milk either pasteurised or unpasteurised is used as a medium for culturing the bacteria. The milk is processed in the vortex and then succussed prior to culture being added If pasteurised milk is used the temperature must reach 72° C. for 15 seconds or more. A starter culture of probiotic bacteria comprising the 8 varieties shown above are added separately. The milk is then incubated between 37° C. and 43° C. to allow growth of more bacteria. During incubation the pH should reach 4.5 to allow the correct balance of beneficial bacteria to be absorbed by the human host consuming the product. The cultures are then dried and capsulated either individually or with additional ingredients. The powder is either capsulated or containerised under an inert gas in airtight containers.

The purpose of administering the dietary composition to patients is to stimulate certain enzymes of the body which when sufficiently active are capable of clearing from the body numerous accumulated undesirable non-end product metabolites and toxins. Sources of such non-end product metabolites and toxins may be environmental, such as exposure to environmental xenobiotic substances—i.e. heavy metals, pesticides, herbicides, fungicides, altered DNA fractions, poisons, certain drugs and pharmaceuticals, as well as excessive levels of other non-end product metabolites which are formed in biochemical reactions in the body during states of altered metabolism—the formulation is able to detoxify infectious organisms such as bacteria, viruses and fungi. All of these may cause oxidative damage to cells.

### EXAMPLE 22

### Asthma Clinical Trial

109 candidates with asthma were selected at random and trialed on the nutrient composition described in Example 1 for a period of 1 month. Over a 4 week period Symptom charts noting frequency of cough, wheeze and shortness of breath were kept by the candidates. Weekly questionnaires denoting drug dosage and frequency of symptoms were also returned to the sponsor. Comparisons of symptoms and drug dosage were made comparing pre and post supplementation with the nutrient composition.

Some of the symptom severities were recorded using fractional values (eg. 0.25) instead of the categories of Nil (0), Mild (1), Moderate (2) and Severe (3). To make use of these entries, the severity values were rounded to the nearest integer using the following scheme:
If 0≦severity<0.5 then severity=0.
If 0.5≦severity<1.5 then severity=1.
If 1.5≦severity<2.5 then severity=2.
If 2.5≦severity<3.0 then severity=3.

The frequency and percentage distributions of the reported bronchodilator use at enrolment and after four weeks of treatment were examined to get an indication of whether a change had occurred.

Cross tabulations of the symptom severities at enrolment and after the four weeks of treatment were performed to describe how the severities had changed and to what degree over this period.

Differences in bronchodilator use before and after the treatment period we compared using paired t tests. The symptom severity values are ordinal variables so the Wilcoxon rank sum test was used to determine whether the baseline and week four symptom severity distributions differed primarily in location. That is whether one of the distributions has been shifted left or right of the other.

One-sided tests of significance were used since it was expected that the treatment would improve the severity of the symptoms and reduce the amount of bronchodilators used by the subjects. All tests of statistical significance were made at the 5% level.

Symptom Severity Cross Tabulations

Coughing

From Table 5 67.9% (74 of 109) subjects had some reduction in the severity of their coughing after four weeks of the treatment, 27.5% (30 of 109) remained the same and 4.6% (5 of 109) got worse. This was likely due to an inadequate daily dose and also the winter influenza outbreak.

Among those who initially had severe coughing after the treatment, 37.1% (13 of 35) did not report any coughing, 37.1% (13 of 35) reported mild coughing, 14.3% (5 of 35) reported coughing of moderate severity and 11.4% (4 of 35) reported no improvement (Table 5).

TABLE 5
CROSS TABULATION OF COUGH SEVERITY AT
ENROLMENT BY COUGH SEVERITY AFTER FOUR
WEEKS OF TREATMENT

| | Cough Severity After Treatment | | | | | | | | |
|---|---|---|---|---|---|---|---|---|---|
| Cough severity | Nil | | Mild | | Moderate | | Severe | | Total |
| at enrolment | N | % | N | % | N | % | N | % | N |
| Nil | 13 | 100 | 0 | 0 | 0 | 0. | 0 | 0.00 | 13 |
| Mild | 13 | 50 | 9 | 34.6 | 2 | 7.7 | 2 | 7.69 | 26 |
| Moderate | 16 | 45.7 | 14 | 40.0 | 4 | 11.4 | 1 | 2.86 | 35 |
| Severe | 13 | 37.1 | 13 | 37.1 | 5 | 14.3 | 4 | 11.43 | 35 |
| Total | 55 | | 36 | | 11 | | 7 | | 109 |

Shortness of Breath

A similar pattern was found for shortness of breath and wheezing.

For shortness of breath, 78.9% (86 of 109) reported a reduction in severity, 18.3% (20 of 109) reported no change and 2.8% (3 of 109) reported getting worse (Table 6).

For those who initially reported having a severe shortness of breath, 28.8% (11 of 41) reported no shortness of breath after four weeks of treatment, 39.0% (16 of 41) had moved to the mild category, 19.5% (8 of 41) were in the moderate category and 14.6% (6 of 41) reported no change (Table 6).

TABLE 6
CROSS TABULATION OF SHORTNESS OF BREATH
SEVERITY AT ENROLMENT BY SHORTNESS OF BREATH
SEVERITY AFTER FOUR WEEKS OF TREATMENT

| Shortness of | | | | | | | | | |
|---|---|---|---|---|---|---|---|---|---|
| breath | Shortness of breath severity after treatment | | | | | | | | |
| severity at | Nil | | Mild | | Moderate | | Severe | | Total |
| enrolment | N | % | N | % | N | % | N | % | N |
| Nil | 3 | 100.00 | 0 | 0.00 | 0 | 0.00 | 0 | 0.00 | 3 |
| Mild | 11 | 57.89 | 5 | 26.32 | 2 | 10.53 | 1 | 5.26 | 19 |
| Moderate | 21 | 45.65 | 18 | 39.13 | 6 | 13.04 | 1 | 2.17 | 46 |
| Severe | 11 | 26.83 | 16 | 39.02 | 8 | 19.51 | 6 | 14.63 | 41 |
| Total | 46 | | 39 | | 16 | | 8 | | 109 |

Wheezing

For the wheezing symptom, 68.8% (75 of 109) showed some improvement in symptoms, 28.4% (31 of 109) did not change and 2.8% (3 of 109) were worse off (Table 7).

For those initially in the severe-wheezing category, 37.5% (12 of 32) reported no wheezing after treatment, 34.4% (11 of 32) were in the mild group, 12.5% (4 of 32) had moved to the moderate group and 15.6% (5 of 32) reported no improvement. (Table 7).

TABLE 7
CROSS TABULATION OF WHEEZE SEVERITY AT
ENROLMENT BY WHEEZE SEVERITY AFTER
FOUR WEEKS OF TREATMENT

| Wheeze | Wheeze severity after treatment | | | | | | | | |
|---|---|---|---|---|---|---|---|---|---|
| severity at | Nil | | Mild | | Moderate | | Severe | | Total |
| enrolment | N | % | N | % | N | % | N | % | N |
| Nil | 12 | 100 | 0 | 0 | 0 | 0 | 0 | 0 | 12 |
| Mild | 13 | 50 | 12 | 46.2 | 1 | 3.9 | 0 | 0 | 26 |
| Moderate | 18 | 46.2 | 17 | 43.6 | 2 | 5.1 | 2 | 5.1 | 39 |
| Severe | 12 | 37.5 | 11 | 34.4 | 4 | 12.5 | 5 | 15.6 | 32 |
| Total | 55 | | 40 | | 7 | | 7 | | 109 |

Bronchodilator t Test

From the paired t tests on the amount of bronchodilators doses used, a significant decrease in the amount of Ventolin taken via puffer (p-value=0.0007) and nebuliser (p-value=0.0176), as well as Seretide (p-value=0.0084) and Flixotide (p-value=0.0400) after the four week treatment period (Table 8).

An examination of the usage data for the other bronchodilators in the data set showed that only a small proportion of the subjects (at most 15%) used these other products/substances. With such small numbers meaningful analyses could not be performed on these other data.

TABLE 8
PAIRED T TEST RESULTS FOR STATISTICALLY SIGNIFICANT
CHANGES IN BRONCHODILATOR USE BETWEEN
ENROLMENT AND AFTER TREATMENT

| Bronchodilator | DF | t Value | Pr > \|t\| |
|---|---|---|---|
| Ventolin | 107 | −3.49 | 0.0007 |
| Ventolin Nebuliser | 108 | −2.41 | 0.0176 |
| Seretide | 108 | −2.69 | 0.0084 |
| Flixotide | 108 | −2.08 | 0.0400 |

*Please note, these values are statistically significant at the 5% level.

Ventolin puffer use fell from a mean of 3.8 doses at enrolment to 1.7 after four weeks of treatment. The use of Seretide, Flixotide and Ventolin via nebuliser also fell after four weeks of treatment by smaller amounts in absolute terms, however, the proportional change was similar (Table 9).

TABLE 9
MEAN AND MEDIAN NUMBER OF DOES OF BRONCHODILATOR
USE BETWEEN ENROLMENT AND AFTER TREATMENT

| | Mean | |
|---|---|---|
| Bronchodilator | (enrolment) | Mean (week 4) |
| Ventolin | 3.8 | 1.7 |
| Ventolin Nebuliser | 0.7 | 0.2 |
| Seretide | 1.0 | 0.6 |
| Flixotide | 0.5 | 0.3 |

Symptom Severity Non-Parametric Tests
Cough

The Wilcoxon tests suggest that one of the distributions has a significantly higher cough severity scores than the other (Norm approx Z=7.5365, p-value<0.0001) (Table 10). Using the information from Table 5 it can be seen that the severities at the time of enrolment were more severe than the values after the four weeks of treatment.

TABLE 10
WILCOXON TWO SAMPLE TEST RESULTS FOR CHANGES IN
COUGH SEVERITY
Wilcoxon Two-Sample Test

| | |
|---|---|
| Statistic | 15320.5 |
| Normal Approximation | |
| Z | 7.5365 |
| One-Sided Pr > Z | <.0001 |
| Two-Sided Pr > \|Z\| | <.0001 |
| t Approximation | |
| One-Sided Pr > Z | <.0001 |
| Two-Sided Pr > \|Z\| | <.0001 |

Z includes a continuity correction of 0.5.

Shortness of Breath

Similarly the Wilcoxon test for shortness of breath indicated that there was a statistically significant difference in the distributions of severities at enrolment and after four weeks for this symptom (Norm approx Z=8.7827, p-value<0.0001) (Table 11). From Table 6 it can be seen that the severities reported at enrolment were more severe than after the treatment period.

TABLE 11
WILCOXON TWO SAMPLE TEST RESULTS FOR CHANGES IN
SHORTNESS OF BREATH SEVERITY
Wilcoxon Two-Sample Test

| | |
|---|---|
| Statistic | 15891.5 |
| Normal Approximation | |
| Z | 8.7827 |
| One-Sided Pr > Z | <.0001 |
| Two-Sided Pr > \|Z\| | <.0001 |
| t Approximation | |
| One-Sided Pr > Z | <.0001 |
| Two-Sided Pr > \|Z\| | <.0001 |

Z includes a continuity correction of 0.5.

Wheeze

There were statistically significant differences in the distribution of severities for wheezing between the initial severities and those recorded after four weeks. With the information from Table 7 it can be seen in Table 12 that there was a statistically significant improvement in the severities of wheezing after four weeks of treatment.

TABLE 12
WILCOXON TWO SAMPLE TEST RESULTS FOR CHANGES IN
COUGH SEVERITY
Wilcoxon Two-Sample Test

| | |
|---|---|
| Statistic | 15492.5 |
| Normal Approximation | |
| Z | 7.928 |
| One-Sided Pr > Z | <.0001 |
| Two-Sided Pr > \|Z\| | <.0001 |
| t Approximation | |
| One-Sided Pr > Z | <.0001 |
| Two-Sided Pr > \|Z\| | <.0001 |

Z includes a continuity correction of 0.5.

Summary

From these data it appeared that the treatment was associated with a statistically significant decrease in the use of Ventolin (puffer and nebuliser), Seretide and Flixotide, and that is also associated with a significant decrease in the severity of coughing, wheezing and shortness of-breath after four weeks of treatment.

### EXAMPLE 23

### Vortex Theory

Studies on fluid dynamics have shown discrepancies. One group have shown no transverse force on a vortex due to normal fluid flow, whereas earlier work found a transverse force proportional to normal fluid velocity uₙ and normal fluid density rₙ. Applicant has linearized the time-independent two-fluid equations about the exact solution for a vortex, and found three solutions that are important in the region far from the vortex. Uniform fluid flow gives rise to the usual fluid Magnus force. Uniform normal fluid flow gives rise to no forces in the linear region, but does not satisfy reasonable boundary conditions at short distances. A logarithmically increasing normal fluid flow gives a viscous force. As in classical hydrodynamics this logarithmic increase must be cut off by non-linear effects at large distances; this gives a viscous force proportional to uₙ/ln (uₙ) and a transverse contribution that goes like to uₙ/ln (uₙ)², even in the absence of an explicit Iordanskii force. In the limit uₙ 0, no transverse force is found, but at non zero uₙ there are important corrections that were not found previously. The Applicant believes that the Magnus force in a superfluid at non zero temperature is an example of a topological relation for which finite-size corrections may be large.

A vortex threads into limited helical channels: 2-dimensional hydrodynamics of an ideal liquid. A vortex in an isotropic liquid produces small amounts of rotons depending on the speed and energy of the vortex. Rotons are second generation tachyons formed in oscillating vortex. This oscillation must be at the fundamental harmonic of this vortex. This vortex must be greater than 100 mm Radius and at the most 2500 mm Radius and velocity to impart 50 to 200 joules per second.

$$\begin{matrix}Mass , Energy , and Speed of a vortex of an \\ Isotropic Fluid \\ \sim {K}_{f}^{d} + {G}_{{t}_{p}^{np}} + {\sum }_{t}^{g} m = 0 & Equation 1 \\ vortex Distortion of an Isotropic fluid \\ {g}^{μ v} {p}_{u} {p}_{v} = 0 , {g}^{00} = 1 , {g}^{0 i} = - {v}_{s}^{i} , {g}^{ik} = - {c}^{2} {δ}^{ik} + {v}_{s}^{i} {v}_{s}^{k} \\ {ds}^{2} = ( 1 - \frac{{v}_{s}^{2}}{{c}^{2}} ) {( dt + \frac{N κ d ϕ}{2 π ( {c}^{2} - {v}_{s}^{2} )} )}^{2} - \frac{{dr}^{2}}{{c}^{2}} - \frac{{dz}^{2}}{{c}^{2}} - \frac{{r}^{2} d {ϕ}^{2}}{{c}^{2} - {v}_{s}^{2}} \\ {σ}_{⊥} = {\int }_{- ∞}^{+ ∞} \frac{ⅆ x}{{v}_{G}} {\int }_{- ∞}^{+ ∞} ⅆ y \frac{\partial {v}_{sy}}{\partial x} = \frac{N κ}{{v}_{G}} & Equation 2 \\ impact energy of the created rotons \\ \frac{\overline{a}}{a} + \frac{\dot{a}}{a} ( x \frac{\dot{b}}{b} + \frac{\dot{c}}{c} ) - y \frac{{\dot{a}}^{2}}{{a}^{2}} + 2 y \frac{\dot{ⅆ}}{ⅆ} ( \frac{\dot{a}}{a} - \frac{\dot{ⅆ}}{ⅆ} ) + x \frac{{a}^{4} - {( {b}^{2} - {c}^{2} )}^{2}}{2 {a}^{2} {b}^{2} {c}^{2}} + y \frac{{a}^{2} + {b}^{2} - {c}^{2}}{{a}^{2} {c}^{2}} = \frac{1}{2} κ {m}^{2} {ϕ}^{2} + κ \frac{ρ}{2} ( 2 - λ ) , \\ \frac{\ddot{b}}{b} + \frac{\dot{b}}{b} ( x \frac{\dot{a}}{a} + \frac{\dot{c}}{c} ) - y \frac{{\dot{b}}^{2}}{{b}^{2}} + 2 y \frac{\dot{ⅆ}}{ⅆ} ( \frac{\dot{b}}{b} - \frac{\dot{ⅆ}}{ⅆ} ) + x \frac{{b}^{4} - {( {a}^{2} - {c}^{2 } )}^{2}}{2 {a}^{2} {b}^{2} {c}^{2}} + y \frac{{a}^{2} + {b}^{2} - {c}^{2}}{{b}^{2} {c}^{2}} = \frac{1}{2} κ {m}^{2} {ϕ}^{2} + κ \frac{ρ}{2} ( 2 - λ ) , & Equation 3 \\ Feidmann and Einsteing Equations of Vortex Formation . \\ {( \frac{\dot{\tilde{R}}}{\tilde{R}} )}^{2} = \frac{1}{6} κ {m}^{2} {ϕ}^{2} + κ \frac{\dot{{ϕ}^{2}}}{6} + κ \frac{\overline{ρ}}{3} + \frac{{\overline{σ}}^{2}}{6} - \\ x \frac{2 {a}^{2} {b}^{2} + 2 {b}^{2} {c}^{2} + 2 {c}^{2} {a}^{2} - {a}^{4} - {b}^{4} - {c}^{4}}{12 {a}^{2} {b}^{2} {c}^{2}} + y \frac{1}{3 {c}^{2}} , \\ \frac{\ddot{\ddot{\tilde{R}}}}{\tilde{R}} = \frac{1}{6} κ {m}^{2} {ϕ}^{2} - κ \frac{\dot{{ϕ}^{2}}}{3} - κ \frac{\overline{ρ}}{3} - \frac{{\overline{σ}}^{2}}{3} , & Equation 4 \\ The velocity of the fluid vortex line {V}_{L} is \\ {V}_{L} = {h}_{1} ( {V}_{s} + {V}_{I} ) + {h}_{2} \hat{t} \times ( {v}_{n} - {V}_{s} - {V}_{I} ) + {h}_{3} {v}_{n} , \\ where \\ {h}_{1} = \frac{{ρ}_{s} κ {D}_{0}}{{D}_{0}^{2} + {D}^{2}} , \\ {h}_{2} = \frac{{\dot{ρ}}_{s} κ D}{{D}_{0}^{2} + {D}^{2}} , \\ {h}_{3} = \frac{{D}^{2} - {D}_{0} {D}_{t}}{{D}_{0}^{2} + {D}^{2}} , \\ {D}_{0} = {ρ}_{s} κ - {D}_{t} . & Equation 5\end{matrix}$$

D and Dₜ are mutual friction coefficients, rₛ is the fluid density, k is the quantum of circulation, Vᵣ is the velocity induced by the presence of any fluid vortex filaments, and Vₛ is any externally applied fluid velocity field.

For a free vortex core, or one bound by a core whose size is much less than the mean free path of excitations, then we should take account of the effect of the superfluid flow on non-interacting excitations, in accordance with the discussions.

According to these works the flow of phonons or rotons past a stationary vortex produces a transverse force equal to Equation 6.

$${F}_{t} = - \frac{{ρ}_{n} h}{m} {z}^{⋒} \times {u}_{c} , \\ {c}_{1} = [ - 1 + \frac{3 {K}_{0} ( a )}{2 {aK}_{1} ( a ) ( 1 - ⅈα )} ] {c}_{3} = - {c}_{2} , \\ {d}_{2} = - \frac{3 {K}_{1}^{′} ( a )}{{aK}_{1} ( a ) ( 1 - ⅈα )} {c}_{3} , \\ {d}_{3} = - \frac{3}{{aK}_{1} ( a ) ( 1 - ⅈα )} {c}_{3} . \qquad Equation 7$$

This machine system uses the kinetic energy of isotropic fluids of a range between 40,000 and 80,000 kJ as a function in the production of thermodynamic rotons and variables such as temperature and pressure has continued. Measurements have shown that in contrast to calculations, the kinetic energy of the solution is significantly higher than expectations. Based on values taken from the rest before processing. These experiments are been extended and refined. A study of fluid dynamics of vortices has shown that current molecular models provide a poor description of the cross-over region between molecular and atomic behaviour. More recent research cites more detailed description of the cross-over region.

$$\begin{matrix}0 = {P}_{μ} ( j ) {P}_{μ} ( j ) {ψ}^{2} ( j ) + {m}^{2} {c}^{4} {ψ}^{2} ( j ) \\ = {δ}_{μ v} {P}_{μ} ( j ) ψ ( j ) {P}_{v} ( j ) ψ ( j ) + {mc}^{2} ψ ( j ) {mc}^{2} ψ ( j ) \\ = ( {δ}_{μ v} + {δ}_{v μ} ) {P}_{μ} ( j ) ψ ( j ) {P}_{v} ( j ) ψ ( j ) ( μ \geq v ) + \\ {mc}^{2} ψ ( j ) {mc}^{2} ψ ( j ) \\ = 2 {δ}_{μ v} {P}_{μ} ( j ) ψ ( j ) {P}_{v} ( j ) ψ ( j ) ( μ \geq v ) + \\ {mc}^{2} ψ ( j ) {mc}^{2} ψ ( j ) \\ = 2 {δ}_{μ v} {δ}_{jk} {δ}_{jl} {P}_{μ} ( k ) ψ ( k ) {P}_{v} ( l ) ψ ( l ) ( μ \geq v ) + \\ {δ}_{jk} {δ}_{jl} {mc}^{2} ψ ( k ) {mc}^{2} ψ ( l )\end{matrix} \qquad Equation 8$$

The energy of the produced rotons is transferred to the fluid in the second stage. This stage used succussion at a rate that is a ratio of frequency x. This frequency x is calculated out by the above formula and Ricci Tensors.

1-25. (canceled)

26. A method of treating a disease in a subject in need of such treatment, comprising the step of administering an effective amount of a substance or active agent which comprises one or more components which have been agitated such that a harmonic of between 20 to 50 Hz has been produced, with the proviso that the disease is not an airway disorder.

27. A method according to claim 26, wherein the disease is selected from the group consisting of arthritis, osteoporosis, tendonitis, fibromyalgia and traumatic injury.

28. A method according to claim 26, wherein the disease is arthritis.

29. A method according to claim 26, wherein the subject is a warm-blooded vertebrate.

30. A method according to claim 29, wherein the warm-blooded vertebrate is a mammal or a bird.

31. A method according to claim 30, wherein the mammal is selected from the group consisting of humans, dogs, cats, swine, ruminants, primates and horses.

32. A method according to claim 26, wherein the substance contains an active agent.

33. A method according to claim 32, wherein the active agent possesses therapeutic or prophylactic properties in vivo.

34. A method according to claim 33, wherein the active agent is a probiotic bacterium, protein, nucleic acid, small molecule or combinations thereof.

35. A method according to claim 34, wherein the active agent is a drug, peptide, protein, carbohydrate, nucleoprotein, mucoprotein, lipoprotein, synthetic polypeptide or protein, or a small molecule linked to a protein, glycoprotein, steroid, nucleic acid, nucleotide, nucleoside, oligonucleotides, gene, lipid, hormone, vitamin, mineral, element or combinations thereof.

36. A method according to claim 35, wherein the active agent further includes an antioxidant, chemotherapeutic agent, steroid, hormone, antibiotic, antiviral, antifungal, antiproliferative agent, antihistamine, anticoagulant, non-steroidal and steroidal anti-inflammatory compound.

37. A method according to claim 26, wherein the harmonic of between 20 to 50 Hz is produced by agitating said substance or active agent.

38. A substance or active agent produced by the process of agitation of a starting substance or agent such that a harmonic of between 20 to 50 Hz is produced.

39. A substance or active agent useful for treating a disease in a subject in need of such treatment, comprising ascorbic acid, magnesium and selenomethionine and a pharmaceutically acceptable carrier, wherein at least one component has been agitated such that a harmonic of between 20 to 50 Hz has been produced, said ascorbic acid, magnesium and selenomethionine being combined in an amount effective to treat said disease.

40. A process for preparing a biomorphogenic medicinal composition which comprises:
(a) providing a substance or active agent according to claim 38;
(b) diluting said substance or active agent using at least one dilution step by addition of a diluent to produce, following or each dilution step, a diluted preparation having a lower concentration of the substance or active agent than the concentration of the substance or active agent in the solution being diluted.

41. A method of producing a formulation or composition of a substance or active agent useful for treating a disease in a subject in need of such treatment, said formulation or composition comprising as components a vitamin, a trace element and probiotic bacteria, said method comprising the step of agitating at least one of said components such that a harmonic of between 20 to 50 Hz is produced.

42. A device for activating a substance or active agent to render it useful for treating a disease in a subject, the device comprising a container and a agitator, wherein said agitator is capable of producing in the substance or active agent a harmonic of between 20 to 50 Hz.

43. A method of activating a substance or active agent to be effective for treating a disease in a subject, comprising the steps of
(a) introducing said substance or active agent into the device of claim 42, and
(b) agitating said substance or active agent such that a harmonic of between 20 to 50 Hz is produced,
thereby rendering said substance or active agent effective for treating the disease.

44. A method according to claim 41, wherein the substance is a foodstuff, a chemical composition, a component of said foodstuff or a component of said chemical composition.

45. A method according to claim 41, wherein the active agent is a therapeutically-active or prophylactically-active chemical composition, herbicide, pesticide, or nutrient.

46. A method according to claim 41, wherein the active agent is a protein, a nucleic acid, a chemical compound, or probiotic bacteria.

47. The method of claim 46 wherein said chemical compound is a vitamin, a mineral, an antibiotic a steroid or a decongestant agent.

48. A method according to claim 43, wherein the active agent is an antioxidant, a chemotherapeutic agent, a steroid, a retinoid, a hormone, an antibiotic, an antiviral, an antifungal, an antiproliferative, an antihistamine, an anticoagulant, an antiphotoaging agent, a melanotropic peptide, or a steroidal or nonsteroidal anti-inflammatory compound.

49. A method according to claim 43, wherein the substance or active agent is a herbicide is selected from the group consisting of 2,4-dichlorophenoxyacetic acid (2,4-D; WEEDAR™); 4-(2,4-dichlorophenoxy)butyric acid (2,4-DB); 3′,4′-dichloropropionanilide (DCPA; (Dacthal™); disodium methylarsonate (DSMA; ARSONATE™); S-ethyl dipropylthiocarbamate (EPTC; EPTAM™. ERADICANE™); 4-chloro-2-methylphenoxy)acetic acid (MCPA; (RHONOX™); 4-(4-chloro-2-methylphenoxy)butanoic acid (MCPB; THISTROL™); monosodium methylarsonate (MSMA; ANSAR™); acetochlor (HARNESS™); acetochlor (SURPASS™); acifluorfen (BLAZER™); alachlor (LASSO™); ametryn (EVIK™); amitrole (AMITROL-T™); asulam (ASULOX™); atrazine (AATREX™); azafenidin (MILESTONE™); benefin (BALAN™); bensulfuron (LONDAX™); bensulide (PREFAR™); bentazon (BASAGRAN™); bromacil (HYVAR-X™); bromoxynil (BUCTRIL™); butylate (SUTAN™); carfentrazone-ethyl (AIM™); chloramben (AMIBEN™); chlorimuron-ethyl (CLASSIC™); chlorpropham (FURLOE™);chlorsulfuron (GLEAN™); clethodim (PRISM™); clethodim (SELECT™); clomazone (COMMAND™); clopyralid (STINGER™); cloransulam (FIRST-RATE™); cyanazine (BLADEX™); cycloate (RO-NEET™); cycloxydim (FOCUS™); desmedipham (BETANEX™); dicamba (BANVEL™); dichlobenil (CASORON™); diclofop (HOELON™); diethatyl (ANTOR™); difenzoquat (AVENGE™); diflufenzopyr (DISTINCT™); dimethenamid (FRONTIER™); diquat (DIQUAT™); diuron (KARMEX™); endothall (DESICATE™); ethalfluralin (CURBIT™); ethalfluralin (SONALAN™); ethametsulfuron (MUSTER™); ethofumesate (NORTRON™); fenoxaprop-ethyl (BUGLE™); fenoxaprop-ethyl (OPTION II™); fluazifop-P (FUSILADE DX™); flucarbazone-sodium (MKH 6562™); flufenacet (AXIOM™); flumetsulam (BROADSTRIKE™); flumiclorac (RESOURCE™); flumioxazin (V-53482™); fluometuron (COTORAN™); fluroxypyr (STARANE™); fomesafen (FLEXSTAR™); fomesafen (REFLEX™); glufosinate (RELY™); glyphosate (ROUNDUP™); halosulfuron (PERMIT, SEMPRA™); haloxyfop (GALANT™); hexazinone (VELPAR™); imazameth (CADRE™); imazamethabenz (ASSERT™); imazamox (RAPTOR™); imazaquin (SCEPTER™); imazethapyr (PURSUIT™); isoxaben (GALLERY™); isoxaflutole (BALANCE™); lactofen (COBRA™); linuron (LOROX™); methazole (PROBE™); metolachlor (DUAL™); metribuzin (LEXONE™); metribuzin (SENCOR™); metsulfuron (ALLY™); molinate (ORDRAN™); napropamide (DEVRINOL™); naptalam (ALANAP™); nicosulfuron (ACCENT™); norflurazon (SOLICAM™); oryzalin (SURFLAN™); oxadiazon (RONSTAR™); oxasulfuron (DYNAM™); oxyfluorfen (GOAL™); paraquat (GRAMOXONE EXTRA™); pebulate (TILLAM™); pelargonic acid (SCYTHE™); pendimethalin (PENTAGON™); pendimethalin (PROWL™); phenmedipham (SPIN-AID™); picloram (TORDON™); primisulfuron (BEACON™); prodiamine (BARRICADE™); prometryn (CAPAROL™); pronamide (KERB™); propachlor (RAMROD™); propanil (STAMPEDE™); prosulfuron (PEAK™);pyrazon (PYRAMIN™); pyridate (LENTAGRANN™); pyridate (TOUGH™); pyrithiobac (STAPLE™); quinclorac (FACET™); quizalofop (ASSURE™); rimsulfuron (MATRIX, SHADEOUT™); sethoxydim (POAST™); siduron (TUPERSAN™); simazine (PRINCEP™); sulfentrazone (AUTHORITY™); sulfometuron (OUST™); sulfosate (TOUCHDOWN™); sulfosulfuron (MON™); tebuthiuron (SPIKE™); terbacil (SINBAR™); thiazopyr (VISOR, MANDATE™); thifensulfuron (PINNACLE™); thiobencarb (BOLERO™); tralkoxydim (ACHEIVE™); triallate (FAR-GO™); triasulfuron (AMBER™); tribenuron (EXPRESS™); triclopyr (GARLON™); triclopyr (GRANDSTAND™); trifluralin (TREFLAN™); triflusulfuron (UPBEET™) and vemolate (VERNAM™).

50. A method according to claim 45, wherein the active agent is a pesticide selected from the group consisting of 1,2-dichloropropane; 1-naphthaleneacetamide; 1-naphthylacetic acid; 2,4,5-trichlorophenoxyacetic (2,4,5-T) acid; a 2,4,5-T amine salt; a 2,4,5-T ester; 4-2,4-dichlorophenoxybutyric acid (2,4-DB); 2,4-DB butoxyethyl ester; 2,4-DB dimethylamine salt (2,4-DB-DMAS); ABAMECTIN™; ACEPHATE™; ACIFLUOREN™; ACIFLUORFEN™; ACROLEIN™; ALACHLOR™; ALDICARB™; ALDOXYCARB™; ALDRIN™; AMETRYN™; AMINOCARB™; AMITRAZ™; AMITROLE™; ANCYMIDOL™; ANILAZINE™; arsenic acid; Asulam-Na; ATRAZINE™; AZIMSULFURON™; AZINPHOS-ME™; BARBAN™; BENALAXYL™; BENDIOCARB™; BENEFIN™; BENODANIL™; BENOMYL™; BENSULFURON ME™; BENSULIDE™; BENTAZON™; BIFENOX™; BIFENTHRIN™; BROMACIL™; Bromoxynil butyrate; BROMOXYNIL™; OCTANOATE™; BUTACHLOR™; Butylate; CAPTAFOL™; CAPTAN™; CARBARYL™; CARBENDAZIM™; CARBOFURAN™; Carbon Disulfide; CARBOPHENOTHION™; CARBOXIN™; CDAA; CHLORAMBEN™; CHLORBROMURON™; CHLORDANE™; chlordimeform; chlordimeform HCl; CHLORETHOXYFOS™; CHLORIDAZON™; CHLOROBENZILATE™; CHLORONEB™; CHLOROPICRIN™; CHLOROTHALONIL™; CHLOROXURON™; CHLORPROPHAM™; CHLORPYRIFOS™; chlorpyrifos-methyl; CHLORSULFURON™; CHLOZOLINATE™; CINMETHYLIN™; CLOFENTEZINE™; CLOMAZONE™; CLOPYRALID™; CRYOLITE™; CYANAZINE™; CYCLOATE™; CYFLUTHRIN™; CYHALOTHRIN™; CYHEXATIN™; CYMOXANIL™; CYPERMETHRIN™; CYROMAZINE™; DAMINOZIDE™; DAZOMET™; DBCP™; DCNA DICLORAN™; DDD™; DDE™; DDT™; DEMETON™; DESMEDIPHAM™; DI-ALLATE™; DIAZINON™; DICAMBA™; DICHLOBENIL™; DICHLONE™; DICHLORMID; DICHLOROPROPENE; DICHLORPROP; DICHLORVOS; DICLOFOP-ME; DICOFOL; DICROTOPHOS; DIELDRIN; DIENOCHLOR; DIFLUBENZURON; DIMETHIPIN; DIMETHIRIMOL; DIMETHOATE; DIMETHYLARSINIC ACID; DINITRAMINE; DINOCAP; DINOSEB; DIOXACARB; DIPROPETRYN; DIQUAT DIBROMIDE; DISULFOTON; DIURON; DNOC; DODINE ACETATE SALT; DSMA; ENDOSULFAN; ENDOTHALL; ENDRIN; EPN; EPTC; ESFENVALERATE; ETHALFLURALIN; ETHEPHON; ETHOFUMESATE; ETHOPROP; ETHYLENE DIBROMIDE; ETRIDIAZOLE; FENAMINOSULF; FENAMIPHOS; FENARIMOL; FENBUTATIN OXIDE; FENFURAM; FENITROTHION; FENOPROP; FENOXAPROP-ET; FENOXYCARB; FENPROPATHRIN; FENSULFOTHION; FENTHION; FENURON; FENVALERATE; FERBAM; FLUAZIFOP-BUTYL; FLUAZIFOP-P-BUTYL; FLUCHLORALIN; FLUCYTHRINATE; FLUMETRALIN; FLUMETSULAM; FLUOMETURON; FLUPYRSULFURON METHYL; FLURIDONE; FLUSILAZOLE; FLUSILAZOLEHTM; FLUSILAZOLE; FOMESAFEN; FONOFOS; FORMETANATE HCL; FOSAMINE AMMONIUM; FOSAMINE AMMONIUM; FOSETYL ALUMINUM; GLUFOSINATE-AMMONIUM; GLYPHOSATE; HALOXYFOP-METHYL; HEPTACHLOR; HEXACHLOROBENZENE; HEXAZINONE; HEXAZINONEhtm; HEXAZINONEtxt; HYDRAMETHYLNON; IMAZALIL;IMAZAPYR ACID; IMAZAQUIN ACID; IMAZETHAPYR; IPRODIONE; ISAZOFOS; ISOFENPHOS; ISOPROPALIN; ISOXABEN; LACTOFEN; LENACIL; LENACILhtm; LENACILtxt; LINDANE; LINURON; MALATHION; maleic hydrazide acid; MANCOZEB; MANEB; MCPA; MCPB; MECOPROP; mefluidide; MEPIQUAT chloride; METALAXYL; metaldehyde; methamidophos; metham sodium; methazole; methiocarb; methomyl; METHOXYCHLOR™; methyl bromide; methyl isothiocyanate; methyl parathion; METIPAM™; METOLACHLOR™; METRIBUZIN™; METSULFURON ME™; MEVINPHOS™; MEXACARBATE™; MIREX™; MOLINATE™; MONOCROTOPHOS™; MONOLINURON™; MONURON™; MSMA™; MYCLOBUTANIL™; NALED™; naphthalene; napropamide; naptalam sodium salt; NEBURON™; NICOSULFURON™; NITRAPYRIN™; NITROFEN™; NORFLURAZON™; ORYZALIN™; OXADIAZON™; OXAMYL™; OXYCARBOXIN™; OXYDEMETON-ME; OXYFLUORFEN; PACLOBUTRAZOL™; PARAQUAT DICHLORIDE™; PARATHION™; PEBULATE™; PENDIMETHALIN™; pentachlorophenol; perfluidone; perimiphos-ethyl; PERMETHRIN™; PHENMEDIPHAM™; PHENTHOATE™; PHORATE™; PHOSALONE™; PHOSMET™; PHOSPHAMIDON™; PICLORAM™; PIPERALIN™; PIRIMICARB™; pirimiphos-methyl; pirimiphos-ethyl; primisulfuron-methyl; prochloraz; procymidone; prodiamine; profenofos; profluralin; PROMECARB™; PROMETON; PROMETRYN; PROPACHLOR; PROPAMOCARB HCl; PROPANIL; PROPARGITE™; PROPAZINE™; PROPHAM; PROPICONAZOLE; PROPOXUR; PROPYZAMIDE™; PYRETHRINS™; PYRITHIOBAC SODIUM; QUINOMETHIONATE™; QUINTOZENE; QUIZALOFOP-ET; RESMETHRIN; RIMSULFURON; ROTENONE™; SECBUMETON; SETHOXYDIM; SIDURON™; SIMAZINE™; SIMETRYN™; sodium chlorate; sulfometuron-Me; sulprofos; TAU-FLUVALINATE™; TCA-sodium; TEBUTHIURON; TEMEPHOS; TERBACIL; TERBUFOS; TERBUTRYN; TETRACHLORVINPHOS™; THIABENDAZOLE™; THIDIAZURON™; THIOBENCARB™; THIODICARB™; THIOPHANATE-ME™; THIRAM™; TOLCLOFOS-METHYL™; TOXAPHENE™; TRALOMETHRIN™; TRIADIMEFON™; TRIADIMENOL; TRIALLATE™; TRIASULFURON™; TRIBUFOS™; TRICHLORFON™; TRICHLORONAT™; TRICLOPYR™; TRICYCLAZOLE™; TRIDEPHANE™; TRIFLUMIZOLE™; TRIFLURALW™; TRIFLUSULFURON METHYL™; TRIFORINE™; TRIMETHACARB™; VINCLOZOLD™; ZINEB™ and ZIRAM™.

51. A method according to claim 44, wherein the foodstuff is a baked good; a breakfast cereal; a candy; a chewing gum; a chocolate product; a gelatin dessert; a dairy product; a vegetable oil, a beverage; a non-dairy shortening product; a non-dairy whitener; a whipping agent; an artificial whipped cream; a cured meat; potato chips or another snack food; processed egg whites; a jelly; an infant formula; a salad dressing or a sandwich spread.
